(19) United States
(12) Patent Application Publication
(10) Pub. No.: US 2011/0091433 A1
(43) Pub. Date: Apr. 21, 2011

(54) TREATMENT USING REPROGRAMMED MATURE ADULT CELLS

(76) Inventor: Ilham Mohamed Saleh Saeed Abuljadayel, London (GB)

(21) Appl. No.: 12/581,362

(22) Filed: Oct. 19, 2009

(51) Int. Cl. A61K 35/12; C12N 5/071; C12N 5/0735; C12N 5/078; C12N 5/0781; C12N 5/0783; C12N 5/077; C12N 5/079; C12N 5/0797; C12N 5/0775; A61P 25/28; A61P 13/12; A61P 11/00; A61P 21/00

(52) U.S. Cl. 424/93.71; 424/93.7; 435/377

(57) ABSTRACT

A method of treating various diseases, disorders, or conditions in patient using reprogrammed cells such as retrodifferentiated, transdifferentiated, or redifferentiated cells. The method comprises obtaining committed cells from the patient, retrodifferentiating the committed cells to obtain retrodifferentiated target cells, and administering the retrodifferentiated cells to the patient. In certain embodiments, the method comprises obtaining committed cells from the patient, transdifferentiating the committed cells to obtain transdifferentiated target cells, and administering the transdifferentiated target cells to the patient. The retrodifferentiated or transdifferentiated target cells repair or replenish tissue or cells in the patient.

### INCORPORATION BY REFERENCE

All documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

A method of treating various diseases, disorders, or conditions in patient using reprogrammed cells such as retrodifferentiated, transdifferentiated, or redifferentiated cells. The method comprises obtaining committed cells from the patient, retrodifferentiating the committed cells to obtain retrodifferentiated target cells, and administering the retrodifferentiated cells to the patient. In certain embodiments, the method comprises obtaining committed cells from the patient, transdifferentiating the committed cells to obtain transdifferentiated target cells, and administering the transdifferentiated target cells to the patient. The retrodifferentiated or transdifferentiated target cells repair or replenish tissue or cells in the patient.

### BACKGROUND OF THE INVENTION

Stem cells are characterized by their ability to renew themselves through mitotic cell division and to differentiate into a diverse range of specialized cell types. The two broad types of mammalian stem cells are embryonic stem cells, which are isolated from the inner cell mass of blastocysts, and adult stem cells, which are found in adult tissues. In a developing embryo, stem cells can differentiate into all of the specialized embryonic tissues. In adult organisms, stem cells and progenitor cells replenish specialized cells and maintain the normal turnover of regenerative organs such as blood, skin or intestinal tissues.

Stem cells are abundant in a developing embryo, although, the quantity of stem cells decreases as development progresses. By contrast, an adult organism contains a limited number of stem cells which are confined to certain body compartments.

The therapeutic applications of stem cells have the potential to alter treatments for multiple diseases or disorders. While some adult stem cell therapies, such as bone marrow transplants, already exist, medical researchers anticipate using stem cells to treat a wider variety of diseases including cancer, Parkinson's disease, spinal cord injuries, amyotrophic lateral sclerosis, multiple sclerosis, and muscle damage, among others. Such therapies may take advantage of the stem cells' capability of differentiating into cell types that are necessary to treat the disease.

However there is uncertainty as to the success in treating such ailments using stem cells, as well as concerns as to the ease by which stem cells may be obtained. For example, haematopoietic stem cells are traditionally extracted by isolation from bone marrow, growth factor mobilized peripheral blood, or cord blood (placenta). Haematopoietic stem cells may also be prepared from embryonic stem (ES) cells, which are extracted from embryos obtained using in vitro fertilization techniques. However, extraction from these sources is cumbersome and sometimes hazardous, and may be challenged by ethical concerns. Further, the number of stem cells that may be obtained from these sources are limited. Moreover, the stem cells may experience difficulty in differentiating into the cells necessary to treat the ailment.

### SUMMARY OF THE INVENTION

The present invention relates to the use of reprogrammed cells to repair tissue or replenish tissue or cells in a patient. For example, the present invention relates to the use of retrodifferentiated cells, obtained from retrodifferentiation of differentiated or committed cells, to repair tissue or replenish tissue or cells in a patient. The present invention also relates to the use of transdifferentiated cells, obtained from transdifferentiation of differentiated or committed cells, to repair tissue or replenish tissue or cells in a patient.

The application is based, in part, on Applicant's discovery that committed or somatic cells obtained from a patient may be reprogrammed to result in cells of different lineages, and these reprogrammed cells can be administered to the patient to repair or replenish tissues or cells. Examples of the process of reprogramming include retrodifferentiation and transdifferentiation.

Therefore, Applicants discovered that committed cells may undergo reprogramming to result in cells of a different lineage. For instance, committed cells may undergo retrodifferentiation to result in retrodifferentiated cells, e.g., cells that are less differentiated, such as pluripotent stem cells, and that these retrodifferentiated cells can be administered to the patient to repair or replenish tissues or cells. As another example, committed cells obtained from a patient may undergo transdifferentiation to result in transdifferentiated cells, e.g., cells of a different lineage than the committed cells, and that these transdifferentiated cells may be administered to the patient to repair or replenish tissues or cells.

The invention encompasses a method of repairing or replenishing tissue or cells of a cell lineage in a patient by administering reprogrammed cells to the patient. In particular, the invention encompasses a method of repairing or replenishing tissue or cells of a cell lineage in a patient, comprising (i) obtaining committed cells, (ii) retrodifferentiating the committed cells to obtain retrodifferentiated target cells, and (iii) administering the retrodifferentiated target cells to the patient, wherein the retrodifferentiated target cells redifferentiate into cells of the cell lineage. These redifferentiated cells may be of the same cell lineage or of a different cell lineage as the committed cells.

The invention also encompasses a method of repairing or replenishing tissue or cells of a cell lineage in a patient, comprising (i) obtaining committed cells, (ii) transdifferentiating the committed cells to obtain transdifferentiated target cells, and (iii) administering the transdifferentiated target cells to the patient.

In some embodiments, the patient may be suffering from a disease, disorder, or condition including, but not limited to, bone marrow failure, haematological conditions, aplastic anemia, beta-thalassemia, diabetes, motor neuron disease, Parkinson's disease, spinal cord injury, muscular dystrophy, kidney disease, multiple sclerosis, congestive heart failure, hepatitis C virus, human immunodeficiency virus, head trauma, spinal cord injuries, lung disease, depression, non-obstructive azoospermia, andropause, menopause and infertility, rejuvenation, scleroderma ulcers, psoriasis, wrinkles, liver cirrhosis, autoimmune disease, alopecia, retinitis pigmentosa, crystalline dystrophy/blindness, diabetes, and infertility. Hence, in some embodiments, the reprogrammed cells may be bone marrow cells that treat aplastic anemia, leukemia, lymphoma, or human immunodeficiency virus in the patient.

In some embodiments, the reprogrammed target cells, such as retrodifferentiated cells, transdifferentiated target cells, or redifferentiated cells, may include, but are not limited to, pluripotent stem cells, pluripotent germ cells, haematopoietic stem cells, neuronal stem cells, epithelial stem cells, mesenchymal stem cells, endodermal and neuroectodermal stem cells, germ cells, extraembryonic, embryonic stem cells, kidney cells, alveolar epithelium cells, endoderm cells, neurons, ectoderm cells, islet cells, acinar cells, oocytes, sperm, haematopoietic cells, hepatocytes, skin/keratinocytes, melanocytes, bone/osteocytes, hair/dermal papilla cells, cartilage/chondrocytes, fats cells/adipocytes, skeletal muscular cells, endothelium cells, cardiac muscle/cardiomyocytes, and tropoblasts.

In certain embodiments, the committed cells are obtained from blood or related tissues including bone marrow. The committed cells may be obtained from whole blood, and/or may be obtained through apheresis. The blood may be mobilized or unmobilized blood. Such committed cells include, but are not limited to, T cells, B cells, eosinophils, basophils, neutrophils, megakaryocytes, monocytes, erythrocytes, granulocytes, mast cells, lymphocytes, leukocytes, platelets, and red blood cells. Alternatively, the committed cells may be obtained from neuronal tissue from the central nervous system or peripheral nervous system, muscle tissue, or epidermis and/or dermis tissue from skin.

In certain embodiments, the committed cells are obtained from the blood or tissue of a patient. In some embodiments, the patient from which the committed cells are obtained, and to which the reprogrammed target cells such as retrodifferentiated target cells or the transdifferentiated target cells are administered, is the same patient.

In some embodiments, the committed cells are retrodifferentiated by contacting the committed cells with an agent. For example, the committed cells may be incubated with the agent. In certain embodiments, the agent engages a receptor that mediates capture, recognition or presentation of an antigen at the surface of the committed cells. The receptor may be an MHC class I antigen or an MHC class II antigen. In some embodiments, the class I antigen is an HLA-A receptor, an HLA-B receptor, an HLA-C receptor, an HLA-E receptor, an HLA-F receptor or an HLA-G receptor and said class II antigen is an HLA-DM receptor, an HLA-DP receptor, an HLA-DQ receptor or an HLA-DR receptor.

In certain embodiments, the agent may be an antibody to the receptor, such as a monoclonal antibody to the receptor. In some embodiments, the antibody is monoclonal antibody CR3/43 or monoclonal antibody TAL 1B5. In further embodiments, the agent modulates MHC gene expression, such as MHC class I⁺ and/or MHC class Il⁺ expression.

In some embodiments, the retrodifferentiated cells may undergo redifferentiation in a separate step. For example, the retrodifferentiated cells may be redifferentiated by contacting the retrodifferentiated cells with growth factors including, but not limited to, basic fibroblast growth factor , epidermal growth factor, granulocyte macrophage colony-stimulating factor, stem cell factor, interleukins-1, -3, -6, and -7, basic fibroblast growth factor, epidermal growth factor, granulocyte-macrophage colony stimulating factor, granulocyte-colony stimulating factor, erythropoietin, stem cell factor, and bone morphogenetic proteins. The resulting redifferentiated target cells may then be administered to a patient.

In embodiments of the invention, the committed cells can be transdifferentiated by culturing the committed cells in particular culture conditions. For example, the committed cells may be cultured in particular types of culture media in conjunction with the retrodifferentiation agents. Examples of these culture media may include Dulbecco's Modified Eagle Medium (DMEM), Iscove's Modified Dulbecco's Medium (IMDM), etc. The tissue culture media may also comprise differentiation promoting agent such as vitamin and/or mineral supplements, hydrocortisone, dexamethasone, β-mercaptoethanol, etc. Furthermore, additional culturing conditions include using chelating agents or antibiotics, culturing at certain temperatures or carbon dioxide or oxygen levels, and culturing in certain vessels. The culture conditions can determine the type of transdifferentiated target cell that results.

One aspect of the invention is thereby a method of obtaining target cells. The method may comprise obtaining committed cells and then reprogramming the committed cells. These processes are as described in this application. In some embodiments, the method may comprise retrodifferentiating committed cells. In other embodiments, the method may comprise transdifferentiating committed cells. In yet other embodiments, the method may comprise retrodifferentiating committed cells, and then redifferentiating the retrodifferentiated cells.

Another aspect of the invention is use of one or more reprogrammed target cells in preparation of a medicament for repairing or replenishing tissue or cells of a cell lineage in a patient, or for treating a disease or tissue injury.

Yet, another aspect of the invention is a method of treating a disease or tissue injury in a patient in need thereof. In certain embodiments, the method comprises obtaining committed cells, reprogramming the committed cells to obtain reprogrammed target cells, and administering reprogrammed target cells to the patient. In some embodiments, the target cells may be reprogrammed via retrodifferentiation, transdifferentiation, and/or redifferentiation. In particular embodiments, the target cells are retrodifferentiated target cells, transdifferentiated target cells, and/or redifferentiated target cells. The proceses of obtaining the committed cells and reprogramming the committed cells are as described in this application.

In some embodiments, the reprogrammed target cells, such as retrodifferentiated target cells, transdifferentiated target cells, or redifferentiated target cells, may be administered via injection, implantation, or infusion. These cells may be administered by parenteral, intramuscular, intravenous, subcutaneous, intraocular, oral, transdermal injection, or injection into spinal fluid. In certain embodiments, the retrodifferentiated target cells or transdifferentiated target cells are administered in a pharmaceutical composition. The pharmaceutical composition may comprise the retrodifferentiated target cells or transdifferentiated target cells, and at least one pharmaceutically acceptable excipient.

One aspect of the invention is the pharmaceutical composition for administering the reprogrammed target cells, such as retrodifferentiated target cells or transdifferentiated target cells. The pharmaceutical composition may comprise one or more types of target cells, and at least one pharmaceutically acceptable carrier. Optionally, the pharmaceutical composition may include adjuvants and/or other excipients suitable for administration into a patient.

Another aspect of the invention is a method of preparing a pharmaceutical composition or medicament comprising (i) obtaining committed cells, (ii) reprogramming the committed cells to obtain reprogrammed target cells, and (iii) optionally, combining the reprogrammed target cells with one or more pharmaceutical excipients. In some embodiments, the reprogrammed target cells are combined with one or more pharmaceutical excipients. In other embodiments, the reprogrammed target cells are not combined with one or more pharmaceutical excipients. The processes of obtaining committed cells and reprogramming the committed cells are as described in this application.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of and “consists essentially of have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawing, in which:

FIG. 1 shows immunophenotyping of aphaeresed mononuclear cells before (top panel) and after (lower panel) induction of reprogramming. Cells were labeled with monoclonal antibodies conjugated to R-phycoerythrin (RPE) Cy-5 or phycoerythrins (PE) (vertical legends) for immunoglobulin G1 (IgG1) isotype control and CD34 or CD19, respectively. Cells were also stained for CD45, CD38, CD61, and IgG1 isotype controls monoclonal antibodies conjugated to fluorescein isothiocyanate (FITC) (horizontal legends). Lower panel show increase in haematopoietic stem cells as depicted by in crease in the relative number of CD34 and CD34CD38-cells accompanied by decrease in leukocytes mature markers such as CD45 and CD 19.

FIG. 2a shows sequential immunophenotyping of peripheral blood samples of a patient with severe aplastic anemia following infusion of autologous HRSC (post Day 1, Day 2, Day 3, Day 6, and Day 14). Cells were labeled with monoclonal antibodies against CD34 and CD45 (2^{nd} horizontal panel), and CD34 and CD38 (3^{rd} horizontal panel). The top horizontal panel shows forward and side scatter is flow cytometry, bone marrow smear, and tehphin section of autologous HRSC. Dayl to Dayl4 shows an increase in cells having large forward and side scatter that is indicative of granulocytes, and Dayl to Day3 shows an increase in the relative number of circulating CD34 haematopoietic stem cells. FIG. 2b shows sequential immunophenotyping of peripheral blood samples of a patient with severe aplastic anemia following infusion of autologous human reprogrammed stem cell (HRSC) (post Day 1, Day 2, Day 3, Day 6, and Day 14). Cells were labeled with monoclonal antibodies against CD34 and CD61 (1^{st} horizontal panel), CD19 and CD3 (2^{nd} panel), and CD33&13 and CD7 (3^{rd} horizontal panel). The FACScan plots show increase in the number of myeloid cells, as depicted by a gradual increase in cells expressing CD33&13 including progenitor cells, which is depicted by an increase in the relative number of cell co-expressing CD33&13 with CD7. Also, there was a gradual increase in the relative number of lymphocytes, as shown by an increase in the relative number of CD 19 and CD3 lymphocytes.

FIG. 3 shows bone marrow analysis of a severe aplastic anaemia patient before and after infusion of autologous HRSC. Bone marrow smear before and after therapy (a and b) shows increase in red blood cells; terphine section before and after therapy (c and d) shows increase in bone marrow cellularity; clonal analysis of bone marrow following infusion of HRSC showing increased growth of colony forming unit megakarocyte (e); colony forming monocyte (f); colony forming granulocyte and macrophage (g); and colony forming myelocyte and erythroid (h); and burst forming erythroid (i).

FIG. 4 shows karyotyping and g-banding of peripheral blood sample of a patient with severe aplastic anemia following 4 years of infusion with autologous HRSC showing no genetic abnormalities.

FIG. 5 shows increase in absolute mean fetal hemoglobin levels in patients with thalassemia, post-treatment with autologous reprogrammed cells.

FIG. 6 shows increase in mean corpuscular volume, which represents the average size of red blood cells, expressed in femtoliters, in patients with thalassemia, post-treatment with autologous reprogrammed cells.

FIG. 7 shows increase in mean cell hemoglobin, which is the weight of the hemoglobin per cell, expressed in picograms, in patients with thalassemia, post-treatment with autologous reprogrammed cells.

FIG. 8 shows decrease in serum ferritin levels, expressed in nanograms per milliliter, in patients with thalassemia, post-treatment with autologous reprogrammed cells.

FIG. 9 shows increase in C-peptide levels fasting and post mixed meal diet intake in patients with diabetes, post-treatment with autologous reprogrammed cells.

FIG. 10 shows decrease in glycosylated hemoglobin (HbA1C) levels in patients with diabetes, post-treatment with autologous reprogrammed cells.

FIG. 11 shows decrease in creatine phosphokinase (CPK) and lactate dehydrogenase (LDH) levels in patients with muscular dystrophy, post-treatment with autologous reprogrammed cells.

FIG. 12 shows decrease in liver enzymes alanine aminotransferase (ALT) and aspartate aminotransferase (AST) levels in patients with muscular dystrophy, post-treatment with autologous reprogrammed cells.

FIG. 13 shows decrease in microalbumin urea levels in 12 patients with kidney disease, post-treatment with autologous reprogrammed cells.

FIG. 14 shows decrease in glycosylated hemoglobin (HbA1C) levels in 12 patients with kidney disease due to diabetes, post-treatment with autologous reprogrammed cells.

FIG. 15a shows magnetic resonance imaging (MRI) scans of the brain of a patient with multiple sclerosis before (left scan) and three months after (right scan) treatment with autologous reprogrammed cells depicting decrease in lesion enhancement post stem cell therapy. FIG. 15b shows MRI scans of a different part of the brain of the patient before (left scan) and three months after (right scan) treatment with autologous reprogrammed cells. In the scans depicting before treatment, the arrows point to a lesion in the brain, while in the scans depicting after treatment, the arrows point to the improvement in the lesion.

FIG. 16a shows magnetic resonance imaging (MRI) scans of the brain of a patient with multiple sclerosis before (top scans) and six months after (bottom scans) treatment with autologous reprogrammed cells. FIG. 16b shows additional MRI scans of the brain of the patient before (top scans) and six months after (bottom scans) treatment with autologous reprogrammed cells. In the scans depicting before treatment, the arrows point to a lesion in the brain, while in the scans depicting after treatment, the arrows point to the improvement in the lesion with reduction in brain atrophy as depicted by reduction in ventricle and sulci dilatation.

FIG. 17a shows sagittal magnetic resonance imaging (MRI) scans of a patient with multiple sclerosis before (left scan) and six months after (right scan) treatment with autologous reprogrammed cells. FIG. 17b shows transverse MRI scans of the spinal cord of the patient before (left scan) and six months after (right scan) treatment with autologous reprogrammed cells. In the scans depicting before treatment, the arrows point to a lesion on the spinal cord, while in the scans depicting after treatment, the arrows point to the improvement in the lesion.

FIG. 18a shows decrease liver enzymes alanine aminotransferase (ALT) levels and FIG. 18b shows decrease in aspartate aminotransferase (AST) levels in patients infected with hepatitis C, post-treatment with autologous reprogrammed cells

FIG. 19a shows magnetic resonance imaging (MRI) scans of the brain of a patient with head trauma due to a motor accident. Before treatment (top scans), the ventricles show dilatation and displacement with a wide speared haematoma. After treatment with autologous reprogrammed cells (bottom scans), the ventricles show decrease in brain atrophy parameters such as reduction in ventricle and sulci dilatation with amelioration of haematoma. FIG. 19b shows additional MRI scans of the brain of the patient before (top scans) and after (bottom scans) treatment.

FIG. 20 shows chest x-rays of a patient with lung disease before (left x-ray) and after (right x-ray) treatment with autologous reprogrammed cells. After treatment, the patient shows improvement in lung volume and reduction in lesion size as depicted by decrease in hypo-dense areas.

FIG. 21 shows sex hormone levels for follicle-stimulating hormone (fsh), luteinizing hormone (1h), progesterone (pro), and testosterone (test) in patients with non-obstructive azoospermia and treated with autologous reprogrammed cells. The levels show significant increase in free testosterone with an increase in testis size (data not shown) determined by ultrasound.

FIG. 22 shows retinal sensitivity and visual impairment in a patient suffering from impaired vision before (top panel) and after (bottom panel) treatment with autologous reprogrammed cells. In the Retinal Sensitivity results, the orange area indicates decreased retinal sensitivity. In the Visual Impairment results, the white areas indicate normal vision and the pink, orange, and black areas indicate increased visual impairment. After treatment, the patient experienced improvement in his visual field, as orange areas in the retinal sensitivity results before treatment turned green and white after treatment, and black areas in the visual impairment results before treatment turned white after treatment.

### DETAILED DESCRIPTION

### Definitions

As used herein, “committed cells” are cells that display a differentiated character. These cells are often considered mature and specialized. Examples include white blood cells, red blood cells, epithelial cells, neurons, and chondrocytes.

As used herein, “uncommitted cells” are cells that do not display a mature differentiated character. These cells are often considered immature and are not specialized. An example of an uncommitted cell is a stem cell, which is an immature cell that is capable of self-renewal (division without limit) and differentiation (specialization).

As used herein, “reprogramming” refers to a process by which a committed cell of a first cell lineage is changed into a cell of a different cell type. This different cell type may be of a different cell lineage. Reprogramming may occur through such processes as retrodifferentiation, transdifferentiation, or redifferentiation.

As used herein, a “reprogrammed cell” is a cell that underwent reprogramming of a committed cell. A reprogrammed cell may include a retrodifferentiated cell, transdifferentiated cell, and/or a redifferentiated cell.

As used herein, “retrodifferentiation” is the process by which a committed cell, i.e., mature, specialized cell, reverts back to a more primitive cell stage. “Retrodifferentiated cell” is a cell that results from retrodifferentiation of a committed cell.

As used herein, “transdifferentiation” is the process by which a committed cell of a first cell lineage is changed into another cell of a different cell type. In some embodiments, transdifferentiation may be a combination of retrodifferentiation and redifferentiation. “Transdifferentiated cell” is a cell that results from transdifferentiation of a committed cell. For example, a committed cell such as a whole blood cell may be transdifferentiated into a neuron.

As used herein, “redifferentiation” refers to the process by which an uncommitted cell or a retrodifferentiated cell differentiates into a more mature, specialized cell. “Redifferentiated cell” refers to a cell that results from redifferentiation of an uncommitted cell or a retrodifferentiated cell. If a redifferentiated cell is obtained through redifferentiation of a retrodifferentiated cell, the redifferentiated cell may be of the same or different lineage as the committed cell that had undergone retrodifferentiation. For example, a committed cell such as a white blood cell may be retrodifferentiated to form a retrodifferentiated cell such as a pluripotent stem cell, and then the retrodifferentiated cell may be redifferentiated to form a lymphocyte, which is of the same lineage as the white blood cell (committed cell), or redifferentiated to form a neuron, which is of a different lineage than the white blood cell (committed cell).

As used herein, “target cell” is a cell that is obtained for administration into a patient to repair or replenish tissue or cells. For instance, a target cell may be a reprogrammed target cell, such as a retrodifferentiated target cell or a transdifferentiated target cell, whereby the retrodifferentiated or transdifferentiated target cell is administered to the patient.

### Committed Cells

As described above, committed cells of the invention are cells that display a differentiated character. The committed cell may comprise any components that are concerned with antigen presentation, capture or recognition. For example, the committed cell may be an MHC Class I⁺ and/or an MHC Class II⁺ cell.

The committed cell may also be any cell derived or derivable from an undifferentiated cell. Thus, in one embodiment, the committed cell is also an undifferentiated cell. By way of example, therefore, the committed cell can be a lymphoid stem cell or a myeloid stem cell, which is differentiated relative to a pluripotent stem cell.

Committed cells may be derived from biological material, such as blood or related tissues including bone marrow or cord blood, neuronal tissue from the central nervous system or peripheral nervous system, muscle tissue, or epidermis and/or dermis tissue from skin (i.e. by way of oral scraping for instance). The biological material may be of post-natal origin.

The biological material may be obtained using methods known in the art that are suitable for the tissue type. Examples include, but are not limited to, excision, needle withdrawal, swabbing, and apheresis.

In particular embodiments, the committed cells are derived from whole blood or processed products thereof, such as plasma or the buffy coat, since their removal from subjects can be carried out with the minimum of medical supervision. Blood samples are typically treated with anticoagulants such as heparin or citrate. Cells in the biological sample may be treated to enrich certain cell types, remove certain cell types or dissociate cells from a tissue mass. Useful methods for purifying and separating cells include centrifugation (such as density gradient centrifugation), flow cytometry and affinity chromatography (such as the use of magnetic beads comprising monoclonal antibodies to cell surface markers or panning) (see Vettese-Dadey, The Scientist 1999, 13: 21). By way of example, Ficoll-Hypaque separation is useful for removing erythrocytes and granulocytes to leave mononuclear cells such as lymphocytes and monocytes.

Examples of committed cells that can be derived from blood include, but are not limited to, CFC-T cells, CFC-B cells, CFC-Eosin cells, CFC-Bas cells, CFC-Bas cells, CFC-GM cells, CFC-M, CFC-MEG cells, BFC-E cells, CFC-E cells, T cells, B cells, eosinophils, basophils, neutrophils, monocytes, megakaryocytes, and erythrocytes.

Blood derived committed cells may be identified by their expression of particular antigens. For instance, B cells are CD 19⁺, CD21⁺, CD22⁺ and DR⁺ cells. T cells are CD2⁺, CD3⁺, and either CD4⁺ or CD8⁺ cells. Immature lymphocytes are CD4⁺ and CD8⁺ cells. Activated T cells are DR⁺ cells. Natural killer cells (NKs) are CD56⁺ and CD16⁺ cells. T lymphocytes are CD7⁺ cells. Leukocytes are CD45⁺ cells. Granulocytes are CD13⁺ and CD33⁺ cells. Monocyte macrophage cells are CD 14⁺ and DR⁺ cells.

In certain embodiments, the committed cell may be a B lymphocyte (activated or non-activated), a T lymphocyte (activated or non-activated), a cell from the macrophage monocyte lineage, a nucleated cell capable of expressing class I or class II antigens, a cell that can be induced to express class I or class II antigens or an enucleated cell (i.e. a cell that does not contain a nucleus—such as a red blood cell).

In alternative embodiments, the committed cell may be selected from any one of a group of cells comprising large granular lymphocytes, null lymphocytes and natural killer cells, each expressing the CD56 and/or CD16 cell surface receptors.

Since the committed cells are essentially primary cultures, it may necessary to supplement populations of cells with suitable nutrients to maintain viability. Suitable culture conditions are known by the skilled person in the art. Nonetheless, treatment of cell populations is preferably initiated as soon as possible after removal of biological samples from patients, typically within 12 hours, preferably within 2 to 4 hours. Cell viability can be checked using well known techniques such as trypan blue exclusion or propidium iodide.

### Retrodifferentiated Cells

Retrodifferentiation is a type of a reprogramming process whereby structures and functions of cells are progressively changed to give rise to less specialized cells. Retrodifferentiation can occur naturally, wherein cells may undergo limited reverse differentiation in vivo in response to tissue damage. Alternatively, retrodifferentiation may be induced using the methods described in U.S. application Ser. No. 08/594,164, now U.S. Pat. No. 6,090,625; U.S. application Ser. No. 09/742,520, now U.S. Pat. No. 7,112,440; U.S. application Ser. No. 10/140,978, now U.S. Pat. No. 7,220,412; U.S. application Ser. No. 10/150,789, now U.S. Pat. No. 7,410,773; and U.S. application Ser. No.09/853,188, which are all incorporated herein by reference.

Retrodifferentiated cells of the invention may include, but are not limited to pluripotent stem cells, lymphoid stem cells, myeloid stem cells, neural stem cells, skeletal muscle satellite cells, epithelial stem cells, endodermal stem cells, mesenchymal stem cells, and embryonic stem cells.

In particular embodiments, the committed cells are derived from blood and are retrodifferentiated to form retrodifferentiated cells of the haematopoietic cell lineage. Examples of these retrodifferentiated cells include, but are not limited to, pluripotent stem cells, lymphoid stem cells, and myeloid stem cells.

Committed cells may be retrodifferentiated by contacting the cells with an agent that operably engages the cells. The cells are then incubated so as to allow those cells that have been operably engaged by the agent to progress through the retrodifferentiation process and ultimately become undifferentiated.

The contacting step may comprise the agent engaging with surface antigens on the committed cell. The agent may act in direct engagement or in indirect engagement with the committed cell. An example of direct engagement is when the committed cell has at least one cell surface receptor on its cell surface, such as a β-chain having homologous regions (regions that are commonly found having the same or a similar sequence) such as those that may be found on B cells, and wherein the agent directly engages the cell surface receptor. Another example, is when the committed cell has a cell surface receptor on its cell surface such as an α-chain having homologous regions such as those that may be found on T cells, and wherein the agent directly engages the cell surface receptor.

An example of indirect engagement is when the committed cell has at least two cell surface receptors on its cell surface and engagement of the agent with one of the receptors affects the other receptor which then induces retrodifferentiation of the committed cell.

The agent for the retrodifferentiating the committed cell may be a chemical compound or composition. For instance, the agent may be capable of engaging a cell surface receptor on the surface of the committed cell. In certain embodiments, the agent operably engages a receptor present on the surface of the committed cell—which receptor may be expressed by the committed cell, such as a receptor that is capable of being expressed by the committed cell.

For example, agents may include, but are not limited to, any one or more of cyclic adenosine monophosphate (cAMP), a CD4 molecule, a CD8 molecule, a part or all of a T-cell receptor, a ligand (fixed or free), a peptide, a T-cell receptor (TCR), an antibody, a cross-reactive antibody, a monoclonal antibody, or a polyclonal antibody. Growth factors may also be used, such as haematopoietic growth factors, for example erythropoietin and granulocyte-monocyte colony stimulating factor (GM-CSF).

If the agent is an antibody, a cross-reactive antibody, a monoclonal antibody, or a polyclonal antibody, then the agent may be any one or more of an antibody, a cross-reactive antibody, a monoclonal antibody, or a polyclonal antibody to any one or more of: the .beta. chain of a MHC class II antigen, the n-chain of a MHC HLA-DR antigen, the α-chain of a MHC class I or class II antigen, the α-chain of HLA-DR antigen, the α-and the β-chain of MHC class II antigen or of a MHC class I antigen. An example of an antibody is CR3/43 (supplied by Dako).

The term “antibody” may include the various fragments (whether derived by proteolytic cleavage or recombinant technology) and derivatives that retain binding activity, such as Fab, F(ab′)₂ and scFv antibodies, as well as mimetics or bioisosteres thereof. Also included as antibodies are genetically engineered variants where some of the amino acid sequences have been modified, for example by replacement of amino acid residues to enhance binding or, where the antibodies have been made in a different species to the organism whose cells it is desired to treat according to the methods of the invention, to decrease the possibility of adverse immune reactions (an example of this is ‘humanized’ mouse monoclonal antibodies).

Agents used to effect the conversion of a committed cell to a retrodifferentiated cell preferably may act extracellularly of the committed cell. For example, the committed cell may comprise a receptor that is operably engageable by the agent and the agent operably engages the receptor.

For example the receptor may be a cell surface receptor. Specific examples of cell surface receptors include, but are not limited to, MHC class I and class II receptors. The receptor may comprise an α-component and/or a β-component, as is the case for MHC class I and class II receptors.

The receptor may comprises a β-chain having homologous regions, for example at least the homologous regions of the β-chain of HLA-DR.

Alternatively, or in addition, the receptor may comprise an α-chain having homologous regions, for example at least the homologous regions of the α-chain of HLA-DR. The receptor may be a Class I or a Class II antigen of the major histocompatibility complex (MHC). In certain embodiments the cell surface receptor may include, but are not limited to, an HLA-DR receptor, a DM receptor, a DP receptor, a DQ receptor, an HLA-A receptor, an HLA-B receptor, an HLA-C receptor, an HLA-E receptor, an HLA-F receptor, or an HLA-G receptor. In some embodiments, the cell surface receptor may be an HLA-DR receptor.

The agent may be an antibody to the receptor, such as a monoclonal antibody to the receptor.

An example of an agent may be one that modulates MHC gene expression such as MHC Class I⁺ and/or MHC Class II⁺ expression.

In certain embodiments, the agent may be used in conjunction with a biological response modifier. Examples of biological response modifiers include, but are not limited to, an alkylating agent, an immunomodulator, a growth factor, a cytokine, a cell surface receptor, a hormone, a nucleic acid, a nucleotide sequence, an antigen or a peptide. For instance, an alkylating agent may be or may comprise cyclophosphoamide.

Other biological response modifiers may include compounds capable of upregulating

MHC class I and/or class II antigen expression, which, in some embodiments, may allow an agent that binds to an MHC receptor to work more effectively.

As any cell type can be made to express MHC class I and/or class II antigens, this may provide a method for retrodifferentiating a wide variety of cell types whether they constitutively express class I and/or class II MHC antigens or not.

Committed cells are generally incubated with an agent for at least two hours, typically between 2 and 24 hours, preferably between 2 and 12 hours. Incubations are typically performed at from about room temperature or for example about 22° C., up to about 37° C., including 33° C. The progress of the retrodifferentiating procedure can be checked periodically by removing a small aliquot of the sample and examining cells using microscopy and/or flow cytometry. Alternatively, the device can comprise tracking means for on-line monitoring the progress of the retrodifferentiating procedure.

In addition to the use of retrodifferentiating agents, the committed cells may be cultured in autologous plasma or serum, or in fetal blood serum or horse serum. Optionally, the committed cells may be cultured with anticoagulants, chelating agents, or antibiotics. The temperature range for incubating the cells may be extended to 18-40° C., and may also include 4-10% CO₂ and/or 10-35% O₂. Furthermore, incubation may occur in blood bags, tissue culture bags, or plastic vessels, which are coated or uncoated.

Certain types of retrodifferentiated cells may be obtained by retrodifferentiating using particular culture conditions. For example, committed cells may be retrodifferentiated into pluripotent cells by culturing the committed cells in Dulbecco's Modified Eagle Medium (DMEM), non-essential amino acids (NEAA), L-glutamine (L-glu), and β-mercaptoethanol (2 βME), in conjunction with the retrodifferentiating agents. The committed cells may also be initially exposed to chelating agents.

As another example, to obtain mesenchymal cells, committed cells may be cultured—in conjunction with retrodifferentiating agent(s)—using DMEM (low glucose) and L-glu, or DMEM (low glucose), L-glu, 2 βME, and NEAA. Further, the antibiotic gentamycin may also be used in the cell culture.

### Trans Differentiated Cells

Transdifferentiated cells are obtained by culturing committed cells with a tissue culture media in conjunction with retrodifferentiating agents. The committed cells thereby undergo transdifferentiation, wherein the committed cells are transformed to cells of another cell type; in some embodiments, the committed cells are transformed to cells of a different lineage.

The type of target cell obtained through transdifferentiation is dependent on the culturing conditions. These conditions vary according to the type of tissue culture media, the presence/absence of various differentiation promoting agents, the presence/absence of different serums, the incubation temperature, the presence/absence of oxygen or carbon dioxide, and the type of container or vessel used for incubation.

Examples of tissue culture media used for transdifferentiation include, but are not limited to, Iscove's Modified Dulbecco's Medium (IMDM), Dulbecco's Modified Eagle Medium (DMEM), Eagle's minimum essential (EME) medium, alpha-minimum essential medium (α-MEM), Roswell Park Memorial Institute (RPMI; site where medium was developed) 1640, Ham-F-12, E199, MCDB, Leibovitz L-15, Williams Medium E, or any commercially formulated tissue culture medium.

Differentiation promoting agents include anticoagulants, chelating agents, and antibiotics. Examples of such agents may be one or more of the following: vitamins and minerals or derivatives thereof, such as A (retinol), B₃, C (ascorbate), ascorbate 2-phosphate, D such as D₂ or D₃, K, retinoic acid, nicotinamide, zinc or zinc compound, and calcium or calcium compounds; natural or synthetic hormones such as hydrocortisone, and dexamethasone; amino acids or derivatives thereof, such as L-glutamine (L-glu), ethylene glycol tetracetic acid (EGTA), proline, and non-essential amino acids (NEAA); compounds or derivatives thereof, such as β-mercaptoethal, dibutyl cyclic adenosine monophosphate (db-cAMP), monothioglycerol (MTG), putrescine, dimethyl sulfoxide (DMSO), hypoxanthine, adenine, forskolin, cilostamide, and 3-isobutyl-l-methylxanthine; nucleosides and analogues thereof, such as 5-azacytidine; acids or salts thereof, such as ascorbic acid, pyruvate, okadic acid, linoleic acid, ethylenediaminetetraacetic acid (EDTA), anticoagulant citrate dextrose formula A (ACDA), disodium EDTA, sodium butyrate, and glycerophosphate; antibiotics or drugs, such as G418, gentamycine, Pentoxifylline (1-(5-oxohexyl)-3,7-dimethylxanthine), and indomethacin; and proteins such as tissue plasminogen activator (TPA).

These differentiation promoting agents may be used to obtain particular types of target cells. For instance, vitamin B₃ may be used to yield acinar cells such as islet cells or hydrocortisone; dexamethasone may be used to yield cells of mesenchymal origin or epithelial origins (e.g. kidney epithelial cells, skin and associated structures such as dermal papilla cells); and β-mercaptoethal may be used to yield ectodermal cells such as neuronal cells, including accessory cells of the CNS.

The culture medium may contain autologous plasma; platelets; serum such as fetal blood sampling; or sera of mammalian origin such as horse serum. Furthermore, the conversion process can occur inside blood bags, scaffolds, tissue culture bags, or plastic tissue culture vessels. The tissue culture vessels may be adherent or non-adherent tissue culture vessels, or may be coated or uncoated with agents such as gelatin, collagen, matrigels or extracellular matrices that either promote adherence or floatation depending on the required type of tissue or specialized cells to be prepared.

Additional culturing conditions include the temperature, which may be between about 10 and about 60° C., or between about 18 and about 40° C.; levels of carbon dioxide (CO₂), which may be between about 0 and about 20%, or about 4 and about 10%; and oxygen (O₂), which may be between about 0 and about 50%, or about 10 and about 35%.

Examples of methods to obtain the target cells and used in conjunction with retrodifferentiating agents are discussed in Table 1.

TABLE 1
Methods obtain various types of target cells in
conjunction with retrodifferentiating agents.

| | Culture Conditions Used in | |
|---|---|---|
| Target Cell | Conjunction with | |
| Type | Retrodifferentiating Agents | Additional Culture Conditions |
| Pluripotent Stem | DMEM, NEAA, L-glu, 2βME | Prior exposure of committed |
| cells or | | cells to chelating or |
| heterogenous | | anticoagulants agents. |
| population of | | For progenitors, withdrawal of |
| pluripotent | | retrodifferentiating agent by |
| progenitor cells | | diluting with culture media only |
| Mesenchymal | DMEM (low glucose), L-glu; or | Gentamycin |
| stem cells | DMEM (low glucose), L-glu, | |
| | 2βME, NEAA | |
| Pluripotent | RPMI 1640, optionally with | GAIC |
| germ cells | NEAA, L-glu, 2βME for | incubation can be at about 30-32° C. |
| Oocytes | oocytes; or | for sperm and about 38-39° C. |
| Sperms | EME medium, retinol, L-glu, | for oocytes. |
| | sodium pyruvate, sodium lactate, | cells can be exposed to chelating |
| | NEAA for oocytes; or | agents such as EDTA and EGT |
| | DMEM, Hams F12, vitamin C, | prior to retrodifferentiation and |
| | vitamin E, Retinoic acid, retinol, | redifferentiation. |
| | pyruvate, optionally with | For sperm, can include addition |
| | Pentoxifylline for sperm; or | of okadic acid, DMSO and zinc |
| | Culture condition for pluripotent | or zinc compound; Gamete 100 |
| | stem cells listed above and with | can be used as a basal medium |
| | additional culture condition | instead |
| | listed in the next column for | For oocytes, can include addition |
| | either sperm or oocytes. | of dp-cAMP, disodium EDTA, |
| | | forskolin, cilostamide and |
| | | hypoxanthine |
| | | For oocytes, Medium 199 can be |
| | | used as a basal medium instead |
| Kidney | DMEM, Hams F12, and | |
| | hydrocortisone, optionally with | |
| | vitamin K; or | |
| | Culture conditions for | |
| | pluripotent stem cells listed | |
| | above, with hydrocortisone | |
| Alveolar | DMEM, NEAA, L-glu, | With antibiotic G418 and |
| epithelium | optionally with 2 βME, | matrigel coated tissue culture |
| Endoderm | nicotinamide; or | vessels |
| | Culture conditions for | |
| | pluripotent stem cells listed | |
| | above, with nicotinamide, | |
| | optionally with dexamethasone, | |
| | retinoic acid, db-cAMP; or | |
| | IMDM, L-glu, ascorbic acid, | |
| | MTG | |
| Neuron | DMEM with Hams F12, NEAA, | |
| Ectoderm | 2βME, optionally with | |
| | putrescine, retinoic acid, L-glu, | |
| | hydrocortisone, ascorbate; or | |
| | Culture conditions for | |
| | pluripotent stem cells listed | |
| | above, optionally with | |
| | putrescine, retinoic acid, | |
| | hydrocortisone, ascorbate | |
| Islet cells | DMEM, Hams F12, vitamin B₃; | |
| Acinar | or | |
| | RPMI 1640 with vitamin B₃; or | |
| | Culture conditions for | |
| | pluripotent stem cells listed | |
| | above, with Vitamin B₃ | |
| | (nicotinamide), optionally with | |
| | dexamethasone | |
| Haematopoietic | IMDM, optionally with | Incubation can be at 33° C. |
| cells | hydrocortisone; or | Incubation can be at room |
| | IMDM, L glutamine and MTG; | temperature for amplifying |
| | or | megakaryocytes in culture |
| | Culture conditions for | differentiated cells can be |
| | pluripotent stem cells listed | exposed to chelating agents prior |
| | above, with MTG substituted for | to conversion in order to amplify |
| | 2βME, optionally with vitamins | for erythroid progenitors in |
| | | culture |
| | | RPMI 1640 can be used as a |
| | | basal medium for enrichment of |
| | | lymphoid progenitors |
| | | Sodium butyrate and/or 5- |
| | | azacytidine can be added to |
| | | culture to promote primitive |
| | | erythroid differentiation |
| Hepatocytes of | DMEM or IMDM or α-minimum | |
| the liver | essential medium, L-glu, | |
| | optionally with dexamethasone, | |
| | L ascorbic acid-2-phosphate and | |
| | nicotinamide; or | |
| | Williams Medium E, sodium | |
| | pyruvate, dexamethasone; or | |
| | Culture conditions for | |
| | pluripotent stem cells listed | |
| | above, with dexamethasone | |
| Skin/ | Hams F12, DMEM (media ratio | Can be with gentamycine as |
| keratinocytes | 3:1), hydrocortisone, L-glu, | antibiotic |
| | optionally with adenine; or | Can incubate at 36.4° C. |
| | E199 or DMEM with L-glu, | |
| | optionally with hydrocortisone | |
| | and adenine; or | |
| | Culture conditions for | |
| | pluripotent stem cells listed | |
| | above, with hydrocortisone, | |
| | optionally with calcium or | |
| | calcium compounds or ascorbate | |
| Melanocytes | MCDB and Leibovitz L-15, | |
| | TPA, optionally with 3-isobutyl- | |
| | 1-methylxanthine; or | |
| | medium 199 and hydrocortisone | |
| Osteocytes/bone | DMEM, β-glycerophosphate, | |
| | dexamethasone, ascorbate and L- | |
| | glu, optionally with vitamin D; | |
| | or | |
| | Culture conditions for | |
| | pluripotent stem cells listed | |
| | above, with glycerophosphate, | |
| | dexamethasone and ascorbate, | |
| | optionally with vitamin D | |
| Dermal Papila | William E medium, L-glu, | |
| cells/hair | hydrocortisone and/or vitamin D, | |
| | adenine and linoleic acid; or | |
| | DMEM, Hams F12 as basal | |
| | medium, L-glu, hydrocortisone | |
| | and/or vitamin D, adenine and | |
| | linoleic acid; or | |
| | Culture conditions for | |
| | pluripotent stem cells listed | |
| | above, hydrocortisone, vitamin | |
| | D, adenine and linoleic acid | |
| Chondrocytes/ | DMEM, pyruvate, ascorbate 2- | |
| cartilage | phosphate, dexamethasone and | |
| | proline; or | |
| | Culture conditions for | |
| | pluripotent stem cells listed | |
| | above, ascorbate 2-phosphate, | |
| | dexamethasone and proline | |
| Adipocytes/fat | DMEM, dexamethasone, and | |
| cells | indomethacin; or | |
| | dexamethasone and | |
| | indomethacin | |
| Skeletal muscles | DMEM, low glucose, optionally | Can be with gentamycin as |
| | with hydrocortisone, | antibiotic |
| | dexamethasone, L-glutamine and | Can be with low serum |
| | sodium pyruvate; or | concentration. |
| | DMEM and Ham F12 or F10 as | Can be with culture vessels with |
| | basal medium, or DMEM and | gelatin |
| | medium 199; or | Can be at a temperature raised to |
| | Culture conditions for | 39° C. |
| | pluripotent stem cells listed | Can include addition of 5- |
| | above, with glucose, | azacytidine |
| | gentamycine and low serum | |
| Blood vessels | DMEM, NEAA and 2 βME; or | |
| (endothelium) | IMDM and dexamethasone | |
| Cardiac muscle/ | 4:1 DMEM and M199 media or | Gelatin coated culture vessels for |
| cardiomyocytes | DMEM (low glucose), L-glu, | full differentiation and |
| | NEAA; or DMEM (low glucose) | autologous serum or platelets |
| | with ascorbic acid and/or | depleted plasma |
| | DMSO; or | Full differentiation can be |
| | Culture conditions for | observed on cover slip slide |
| | pluripotent stem cells listed | |
| | above with anti-gravity culturing | |
| | or in a vibrating environment | |
| Tropoblast | DMEM, L-glu, 2 βME, NEAA | Can be used to produce |
| | with continuous dilution with the | autologous growth factors and |
| | same culture medium minus the | hormones necessary for |
| | retrodifferentiating agent; or | differentiating cells into |
| | RPMI 1640, 2 βME, sodium | mesoderm and ectoderm, |
| | pyruvate and L-glutamine | including germ stem cells and |
| | | progenitors |

Notably, for the culture conditions described for each cell type in Table 1, withdrawal of the retrodifferentiation agent by subsequent dilution of the culture condition with the corresponding culture medium results in more enhanced transdifferentiation.

During culturing, the culture medium optionally containing the various differentiation promoting agents may be diluted by the addition of more medium but without the retrodifferentiating agent. Not to be bound by theory, dilution seems to increase differentiation because cells become less dense and proliferation stimulating factors are less concentrated. Thus, the addition of medium may further enhance transdifferentiation and will affect what type of cell within the cell lineage is obtained. For instance, if the target cell is a neuron, the addition of culture medium may result in a shift in development towards a more mature neuron rather than a neuron progenitor (both are of the same lineage). As another example, forward differentiation skeletal muscle progenitors will only differentiate by consecutive dilution of culture medium which is achieved by gradually decreasing the serum concentration.

### Redifferentiated Cells

The retrodifferentiated cells may be used to obtain target cells by recommitting or redifferentiating the retrodifferentiated cells to a target cell type. This may be performed by contacting the retrodifferentiated cells with growth factors. For example, retinoic acid has been used to differentiate stem cells into neuronal cells. Methylcellulose followed by co-culture with a bone marrow stromal line and IL-7 has been used to differentiate stem cells into lymphocyte precursors (Nisitani et al., Int Immuno 1994, 6: 909-916). Le Page (New Scientist Dec. 16, 2000) teaches that stem cells can be differentiated into lung epithelial cells. Bischoff (Dev Biol 1986, 115: 129-39) teaches how to differentiate muscle satellite cells into mature muscle fibres. Neural precursor cells can be expanded with basic fibroblast growth factor and epidermal growth factor (Nakafuku and Nakamura, J Neurosci Res 1995, 41: 153-168). Haematopoietic stem cells can be expanded using a number of growth factors including GM-CSF, erythropoietin, stem cell factor and interleukins (IL-1, IL-3, IL-6)—see Metcalf (Nature 1989, 339: 27-30) for a review of these various factors.

Potocnik et al. (EMBO J 1994, 13: 5274-83) even demonstrated the differentiation of stem cells to haematopoietic cells using low oxygen (5%) conditions.

The redifferentiated cell may be of the same lineage as the committed cell from which the retrodifferentiated cell was derived. Alternatively, the redifferentiated cell may be of a different lineage than the committed cell from which the retrodifferentiated cell was derived. For example, a B lymphocyte may be retrodifferentiated to a CD34⁺CD38⁻HLA-DR⁻stem cell. This stem cell may be subsequently redifferentiated or recommitted along a B cell lineage (the same lineage) or a lymphoid lineage (different lineage).

### Target Cells

The target cells of the invention are reprogrammed cells that can be obtained by retrodifferentiation, transdifferentiation, or redifferentiation as described above. According to the invention, target cells may include, but are not limited to, pluripotent stem cells, lymphoid stem cells, myeloid stem cells, neural stem cells, skeletal muscle satellite cells, epithelial stem cells, endodermal and neuroectodermal stem cells, germ cells, extraembryonic and embryonic stem cells, mesenchymal stem cells, kidney cells, alveolar epithelium cells, endoderm cells, neurons, ectoderm cells, islet cells, acinar cells, oocytes, sperm, haematopoietic cells, hepatocytes, skin/keratinocytes, melanocytes, bone/osteocytes, hair/dermal papilla cells, cartilage/chondrocytes, fats cells/adipocytes, skeletal muscular cells, endothelium cells, cardiac muscle/cardiomyocytes, and tropoblasts.

As described above, committed cells and/or retrodifferentiated cells are cultured under particular conditions to induce retrodifferentiation and/or transdifferentiation and/or redifferentiation and obtain the target cells. The duration for which the committed cells and/or retrodifferentiated cells are cultured is not controlled by a particular length of time, but rather by a determination that the target cells have been produced.

The determination of the production of, or the changes in the number of, retrodifferentiated, transdifferentiated, or redifferentiated target cells may be performed by monitoring changes in the relative number of committed cells that downregulates expression of lineage-associated markers or transcription factors, and/or changes in the relative number of cells having cell surface markers characteristic of the target cells. Alternatively, or in addition, decreases in the numbers of cells having cell surface markers typical of the committed cells and not the target cells may be monitored. For example, the target cell may be an embryonic stem cell, which are characterized by many stage-specific markers such as POU5F1 (OCT-4), TERT, KLF4, UTF1, SOX2, Nanog or stage-specific embryonic markers 3 and 4 (SSEA-3 and SSEA-4), high molecular weight glycoproteins TRA-1-60 and TRA-1-81 and alkaline phosphatase (Andrews et al., Hybridoma 1984, 3: 347-361; Kannagi et al., EMBO J 1983, 2: 2355-2361; Fox et al., Dev Biol 1984, 103: 263-266; Ozawa et al., Cell Differ 1985, 16: 169-173). They also do not express SSEA-1, the presence of which is an indicator of differentiation. Other markers are known for other types of stem cells, such as Nestein for neuroepithelial stem cells (J Neurosci 985, 5: 3310). Mesenchymal stem cells are positive for SH2, SH3, CD29, CD44, CD71, CD90, CD106, CD120a and CD124, for example, and negative for CD34, CD45 and CD14. Pluripotent stem cells are CD34⁺DR⁻TdT⁻ cells (other useful markers being CD38⁻ and CD36⁺). Lymphoid stem cells are DR⁺, CD34⁺ and TdT⁺ cells (also CD38⁺). Myeloid stem cells are CD34⁺, DR⁺, CD13⁺, CD33⁺, CD7⁺ and TdT⁺ cells.

Additional cell markers for target cells may be discovered through microarray analysis. The analysis may involve isolating RNA from target cells that were retrodifferentiated and/or transdifferentiated and/or redifferentiated, labeling the isolated RNA with a dye, and hybridizing the isolated RNA to a microarray. The microarray may comprise genes or oligonucleotides representing a whole genome, or may comprise genes or oligonucleotides directed to a particular organ system, tissue system, disease, pathology, etc. Cell markers may be identified by which genes/oligonucleotides exhibit high signal intensity, and are thereby upregulated or downregulated in the target cells. This information can then be applied to determining target cells based on the presence of markers, or even groups or a pattern of markers, that were identified by the microarray analysis.

Confirmation of target cells can also be performed using a number of in vitro assays such as CFC assays (see also, the examples). Very primitive haematopoietic stem cells are often measured using the long-term culture initiating cell (LTC-IC) assay (Eaves et al, J Tiss Cult Meth 1991, 13: 55-62). LTC-ICs sustain haemopoiesis for 5 to 12 weeks.

For other cell types such as cells of the central nervous system, pancreas, liver, kidney, skin, etc., cell culturing may continue until the target cells emerge as characterized by immunohistochemistry, flow cytometry, microarrays, or reverse-transcription polymerase chain reaction (RT-PCR), which are techniques known in the art. This may also include functional assays, for example, engrafting an immunodeficient host or correcting or ameliorating an underlying clinical condition, as observed herein.

The target cells can be identified by microarray or RT-PCR that show acquisition of new lineage-specific transcription factors, proteins, and signals in the target cells. For example, retrodifferentiated stem cells converted to target cells of the ectodermal lineage may express genes such as Nestin, Criptol, isll, LHX1, and/or EN1, and if further differentiated into neurons, will be expressing neurofilaments (NF). On the other hand, cells converted into target cells of the endodermal lineage may express genes such as sox7, sox17, Nodal, PDX1, and/or FOXA2; but target cells further differentiated towards pancreatic islet cells may express genes such as insulin (INS) and neurog3 (NGN3). Notably, the conversion towards the desired target cells may be accompanied by down regulation of mature transcription factors associated with the original starting population that has undergone conversion.

In addition, the determination of the production of target cells may occur by recognizing particular structural and/or morphological characteristics of the target cells, e.g., cell shape, size, etc. These characteristics are known in the art for the target cells of the invention.

Once the relative numbers of the desired cell type have increased to a suitable level, which may for example be as low as 0.1% or as high as 5%, the resulting altered cell populations may be used in a number of ways. With respect to the numbers of target cells, e.g., pluripotent stem cells, formed, it is important to appreciate the proliferative ability of stem cells. Although under some circumstance, the numbers of stem cells or other retrodifferentiated cells formed may appear to be low, studies have shown that only 50 pluripotent haematopoietic stem cells can reconstitute an entire haematopoietic system in a donor mouse. Thus therapeutic utility does not require the formation of a large number of cells.

Conversion of committed cells to retrodifferentiated, transdifferentiated, or redifferentiated target cells may also be carried out in vivo by administration of the agent, admixed with a pharmaceutically carrier or diluent, to a patient. However it is preferred in many cases that retrodifferentiating, transdifferentiating, or redifferentiating is performed in vitro/ex vivo.

Treated populations of cells obtained in vitro may be used subsequently with minimal processing. For example they may be simply combined with a pharmaceutically acceptable carrier or diluent and administered to a patient in need of stem cells.

It may however be desirable to enrich the cell population for the retrodifferentiated, transdifferentiated, or redifferentiated target cells or purify the cells from the cell population. This can conveniently be performed using a number of methods (see Vattese-Dadey—The Scientist 1999, 13). For example cells may be purified on the basis of cell surface markers using chromatography and/or flow cytometry. Nonetheless, it will often be neither necessary nor desirable to extensively purify retrodifferentiated, transdifferentiated, or redifferentiated target cells from the cell population since other cells present in the population (for example stromal cells) may maintain stem cell viability and function.

Flow cytometry is a well-established, reliable and powerful technique for characterizing cells within mixed populations as well as for sorting cells. Thus, the purification or isolation means may comprise a flow cytometer. Flow cytometry operates on the basis of physical characteristics of particles in liquid suspension, which can be distinguished when interrogated with a beam of light. Such particles may of course be cells. Physical characteristics include cell size and structure or, as has become very popular in recent years, cell surface markers bound by monoclonal antibodies conjugated to fluorescent molecules.

Kreisseg et al. (J Hematother 1994, 3: 263-89) state, “[b]ecause of the availability of anti-CD34 monoclonal antibodies, multiparameter flow cytometry has become the tool of choice for determination of haematopoietic stem and progenitor cells.” Kreisseg further describes general techniques for quantization and characterization of CD34-expressing cells by flow cytometry. Further, Korbling et al., (Bone Marrow Transplant 1994, 13: 649-54) teaches purification of CD34⁺ cells by immunoadsorption followed by flow cytometry based on HLA-DR expression. As discussed above, CD34⁺ is a useful marker in connection with stem cells/progenitor cells. Flow cytometry techniques for sorting stem cells based on other physical characteristics are also available. For example, Visser et al. (Blood Cells 1980, 6:391-407) teach that stem cells may be isolated on the basis of their size and degree of structuredness. Grogan et al. (Blood Cells 1980, 6: 625-44) also teach that “viable stem cells may be sorted from simple haematopoietic tissues in high and verifiable purity”.

As well as selecting for cells on the basis of the presence of a cell surface marker or other physical property (positive selection), cell populations may be enriched, purified using negative criteria. For example, cells that possess lineage specific markers such as CD4, CD8, CD42 and CD3 may be removed from the cell population by flow cytometry or affinity chromatography.

A very useful technique for purifying cells involves the use of antibodies or other affinity ligands linked to magnetic beads. The beads are incubated with the cell population and cells that have a cell surface marker, such as CD34, to which the affinity ligand binds are captured. The sample tube containing the cells is placed in a magnetic sample concentrator where the beads are attracted to the sides of the tube. After one or more wash stages, the cells of interest have been partially or substantially completely purified from other cells. When used in a negative selection format, instead of washing cells bound to the beads by discarding the liquid phase, the liquid phase is kept and consequently, the cells bound to the beads are effectively removed from the cell population.

These affinity ligand-based purification methods can be used with any cell type for which suitable markers have been characterized or may be characterized. Urbankova et al. (J Chromatogr B Biomed Appl 1996, 687: 449-52) teaches the micropreparation of haematopoietic stem cells from a mouse bone marrow suspension by gravitational field-flow fractionation. Urbankova et al. further comments that the method was used for the characterization of stem cells from mouse bone marrow because these cells are bigger than the other cells in bone marrow and it is therefore possible to separate them from the mixture. Thus physical parameters other than cell surface markers may be used to purify/enrich for stem cells.

Cell populations comprising reprogrammed target cells such as retrodifferentiated, transdifferentiated, or redifferentiated target cells, and/or purified reprogrammed target cells, such as retrodifferentiated, transdifferentiated, or redifferentiated target cells produced by the methods of the invention, may be maintained in vitro using known techniques. Typically, minimal growth media such as Hanks, RPMI 1640, Dulbecco's Minimal Essential Media (DMEM) or Iscove's Modified Dulbecco Medium, are used, supplemented with mammalian serum such as FBS, and optionally autologous plasma, to provide a suitable growth environment for the cells. Stem cells may be cultured on feeder layers such as layers of stromal cells (see Deryugina et al. Crit Rev Immunology 1993, 13: 115-150). Stromal cells are believed to secrete factors that maintain progenitor cells in an undifferentiated state. A long term culture system for stem cells is described by Dexter et al. (J Cell Physiol 1977, 91: 335) and Dexter et al. (Acta Haematol 1979, 62: 299).

For instance, Lebkowski et al. (Transplantation 1992, 53: 1011-9) teaches that human CD34⁺ haematopoietic cells can be purified using a technology based on the use of monoclonal antibodies that are covalently immobilized on polystyrene surfaces and that the CD34⁺ cells purified by this process can be maintained with greater than 85% viability. Lebkowski et al., (J Hematother 1993, 2: 339-42) also teaches how to isolate and culture human CD34⁺ cells. See also Haylock et al. (Immunomethods 1994, 5: 217-25) for a review of various methods.

Cell populations comprising stem cells and purified preparations comprising stem cells may be frozen/cryopreserved for future use. Suitable techniques for freezing cells and subsequently reviving them are known in the art.

In one aspect, the retrodifferentiating, transdifferentiating, or redifferentiating occurs to cells from or in buffy coat blood samples. The term “buffy coat” means the layer of white cells that forms between the layer of red cells and the plasma when unclotted blood is centrifuged or allowed to stand.

### Methods of Treatment

Reprogrammed target cells of the present invention, such as retrodifferentiated target cells, transdifferentiated target cells, and redifferentiated target cells, may be combined with various components to produce compositions of the invention. The compositions may be combined with one or more pharmaceutically acceptable carriers or diluents to produce a pharmaceutical composition (which may be for human or animal use). Suitable carriers and diluents include, but are not limited to, isotonic saline solutions, for example phosphate-buffered saline. The composition of the invention may be administered by direct injection. The composition may be formulated for parenteral, intramuscular, intravenous, subcutaneous, intraocular, oral, transdermal administration, or injection into the spinal fluid.

Compositions comprising target cells may be delivered by injection or implantation. Cells may be delivered in suspension or embedded in a support matrix such as natural and/or synthetic biodegradable matrices. Natural matrices include, but are not limited to, collagen matrices. Synthetic biodegradable matrices include, but are not limited to, polyanhydrides and polylactic acid. These matrices may provide support for fragile cells in vivo.

The compositions may also comprise the retrodifferentiated or transdifferentiated or redifferentiated target cells of the present invention, and at least one pharmaceutically acceptable excipient, carrier, or vehicle.

Delivery may also be by controlled delivery, i.e., delivered over a period of time which may be from several minutes to several hours or days. Delivery may be systemic (for example by intravenous injection) or directed to a particular site of interest. Cells may be introduced in vivo using liposomal transfer.

Target cells may be administered in doses of from 1×10⁵ to 1×10⁷ cells per kg. For example a 70 kg patient may be administered 14×10⁶ CD34⁺ cells for reconstitution of tissues. The dosages may be any combination of the target cells listed in this application.

The methods of the invention can be used to treat a variety of diseases, conditions, or disorders. Such conditions include, but are not limited to, bone marrow failure, haematological conditions, aplastic anemia, beta-thalassemia, diabetes, motor neuron disease, Parkinson's disease, spinal cord injury, muscular dystrophy, kidney disease, liver disease, multiple sclerosis, congestive heart failure, hepatitis C virus, human immunodeficiency virus, head trauma, lung disease, depression, non-obstructive azoospermia, andropause, menopause and infertility, rejuvenation, scleroderma ulcers, psoriasis, wrinkles, liver cirrhosis, autoimmune disease, alopecia, retinitis pigmentosa, and crystalline dystrophy/blindness or any disorder associated with tissue degeneration.

Aplastic anemia is a rare but fatal bone marrow disorder, marked by pancytopaenia and hypocellular bone marrow (Young et al. Blood 2006, 108: 2509-2519). The disorder may be caused by an immune-mediated pathophysiology with activated type I cytotoxic T cells expressing Thl cytokine, especially y-interferon targeted towards the haematopoietic stem cell compartment, leading to bone marrow failure and hence anhaematoposis (Bacigalupo et al. Hematology 2007, 23-28). The majority of aplastic anaemia patients can be treated with stem cell transplantation obtained from HLA-matched siblings (Locasciulli et al. Haematologica. 2007; 92:11-18.), though, extending this approach to older patients or those that lack family donors remain a great challenge. Despite the reasonable survival rate after HLA-matched allogenic stem cell transplantation, the procedure carries some potential risks due to the immunosuppressive regime used to prevent graft versus host disease (GVDH). For example high dose cyclophosphoamide with or without antithymocyte globulin (ATG) leads to prolonged period of immunosuppression and predisposes the patient to opportunistic infection. Other potential risk is graft failure which may ensue weeks or months after stem cell transplantation (Gottdiener et al., Arch Intern Med 1981, 141: 758-763; Sanders et al. Semin Hematol 1991, 28: 244-249). Moreover the risk of graft failure increases with the number of blood transfusion received prior stem cell transplantation.

Thalassaemia is an inherited autosomal recessive blood disease marked by a reduced synthesis rate of one of the globin chains that make up hemoglobin. Thus, there is an underproduction of normal globin proteins, often due to mutations in regulatory genes, which results in formation of abnormal hemoglobin molecules, causing anemia. Different types of thalassemia include alpha thalassemia, beta thalassemia, and delta thalassemia, which affect production of the alpha globin, beta globin, and delta globin, respectively. Treatments include chronic blood transfusion, iron chelation, splenectomy, and allogeneic haematopoietic transplantation. However, chronic blood transfusion is not available to most patients due to the lack of an HLA-matched bone marrow donor, while allogeneic hematopoietic transplantation is associated with many possible complications such as infections and graft-versus-host disease.

Diabetes is a syndrome resulting in abnormally high blood sugar levels (hyperglycemia). Diabetes refers to a group of diseases that lead to high blood glucose levels due to defects in either insulin secretion or insulin action in the body. Diabetes is typically separated into two types: type 1 diabetes, marked by a diminished production of insulin, or type 2 diabetes, marked by a resistance to the effects of insulin. Both types lead to hyperglycemia, which largely causes the symptoms generally associated with diabetes, e.g., excessive urine production, resulting compensatory thirst and increased fluid intake, blurred vision, unexplained weight loss, lethargy, and changes in energy metabolism. Diabetes is considered to be a chronic disease, without a cure. Treatment options are limited to insulin injections, exercise, proper diet, or, for patients who have type 2 diabetes, some medications, e.g., those that promote insulin secretion by the pancreas, decrease glucose produced by the liver, increase sensitivity of cells to insulin, etc.

Motor neuron diseases refer to a group of neurological disorders that affect motor neurons. Such diseases include amyotrophic lateral sclerosis (ALS), primary lateral sclerosis (PLS), and progressive muscular atrophy (PMA). ALS is marked by degeneration of both the upper and lower motor neurons, which ceases messages to the muscles and results in their weakening and eventual atrophy. PLS is a rare motor neuron disease affecting upper motor neurons only, which causes difficulties with balance, weakness and stiffness in legs, spasticity, and speech problems. PMA is a subtype of ALS that affects only the lower motor neurons, which can cause muscular atrophy, fasciculations, and weakness. There are no known cures for motor neuron diseases. Riluzole, which is believed to reduce damage to motor neurons, has been has approved as a medication for ALS, although it slows down the progression of ALS rather than improves its effects. For PLS, treatments only address symptoms, such as baclofen which can reduce spasticity or quinine which may decrease cramps.

Parkinson's disease (PD) is a neurodegenerative disorder marked by the loss of the nigrostriatal pathway, resulting from degeneration of dopaminergic neurons within the substantia nigra. The cause of PD is not known, but is associated with the progressive death of dopaminergic (tyrosine hydroxylase (TH) positive) mesencephalic neurons, inducing motor impairment. Hence, PD is characterized by muscle rigidity, tremor, bradykinesia, and potentially akinesia. Thus, there is currently no satisfactory cure for Parkinson's disease or treatments for preventing or treating Parkinson's disease or its symptoms. Symptomatic treatment of the disease-associated motor impairments involves oral administration of dihydroxyphenylalanine (L-DOPA), which can lead to a substantial improvement of motor function, but its effects are reduced as the degeneration of dopaminergic neurons progresses. Alternative strategies include neural grafting, which is based on the idea that dopamine supplied from cells implanted into the striatum can substitute for lost nigrostriatal cells, and gene therapy, which can be used to replace dopamine in the affected striatum by introducing the enzymes responsible for L-DOPA or dopamine synthesis such as by introducing potential neuroprotective molecules that may either prevent the TH-positive neurons from dying or stimulate regeneration and functional recovery in the damaged nigrostriatal system.

Spinal cord injury is characterized by damage to the spinal cord and, in particular, the nerve fibers, resulting in impairment of part or all muscles or nerves below the injury site. Such damage may occur through trauma to the spine that fractures, dislocates, crushes, or compresses one or more of the vertebrae, or through nontraumatic injuries caused by arthritis, cancer, inflammation, or disk degeneration. While treatments following spinal cord injury may involve medications such as methylprednisolone, which is a corticosteroid that reduce damage to nerve sells and decreases inflammation in the injured area, or medications that control pain and muscle spasticity, as well as immobilization of the spine or surgery to remove herniated disks or any objects that may be damaging the spine, there is no known means to reverse the damage to the spinal cord.

Muscular dystrophy (MD) refers to a set of hereditary muscle diseases that weaken skeletal muscles. MD may be characterized by progressive muscle weakness, defects in muscle proteins, muscle cell apoptosis, and tissue atrophy. There are over 100 diseases which exhibit MD characteristics, although nine diseases in particular—Duchenne, Becker, limb girdle, congenital, facioscapulohumeral, myotonic, oculopharyngeal, distal, and Emery-Dreifuss—are classified as MD. There are no known cures for MD, nor are there any specific treatments. Physical therapy may maintain muscle tone and surgery may be used to improve quality of life. Further, symptoms such as myotonia may be treated with medications, but there are no long term treatments.

Kidney disease refers to conditions that damage the kidneys and decrease their ability to function, which includes removal of wastes and excess water fro the blood, regulation of electrolytes, blood pressure, acid-base balance, and reabsorption of glucose and amino acids. The two main causes of kidney disease are diabetes and high blood pressure, although other causes include glomerulonephritis, lupus, and malformations and obstructions in the kidney. There is no cure for kidney disease, and thereby therapy focuses on slowing the progression of the disease and treating the causes of the disease, such as through controlling blood glucose and high blood pressure and monitoring diet; treating complications of the disease, for example, by addressing fluid retention, anemia, bone disease; and replacing lost kidney function, such as through dialysis or transplantation.

Multiple sclerosis is an autoimmune condition in which the immune system attacks the central nervous system, leading to demyelination. MS affects the ability of nerve cells in the brain and spinal cord to communicate with each other, as the body's own immune system attacks and damages the myelin which enwraps the neuron axons. When myelin is lost, the axons can no longer effectively conduct signals. This can lead to various neurological symptoms which usually progresses into physical and cognitive disability. There is no known cure for MS; treatments attempt to return function after an attack (sudden onset or worsening of MS symptoms), prevent new attacks, and prevent disability. For example, treatment with corticosteroids may help end the attack, while treatment with interferon during an initial attack has been shown to decrease the chance that clinical MS will develop.

Human immunodeficiency virus (HIV) is a lentivirus that can lead to acquired immunodeficiency syndrome (AIDS), a condition wherein the immune system begins to fail. HIV primarily infects vital cells in the human immune system such as helper T cells, macrophages, and dendritic cells. HIV infection leads to low levels of CD4⁺ T cells by direct viral killing of infected cells, by increased rates of apoptosis in infected cells, or by killing of infected CD4⁺T cells by CD8 cytotoxic lymphocytes that recognize infected cells. Currently, there is no vaccine or cure for HIV or AIDS. Treatment for HIV infection consists of highly active antiretroviral therapy, or HAART. Current HAART options are combinations (or “cocktails”) consisting of at least three drugs of at least two types antiretroviral agents. Typically, these classes are two nucleoside analogue reverse transcriptase inhibitors (NARTIs or NRTIs) plus either a protease inhibitor or a non-nucleoside reverse transcriptase inhibitor (NNRTI).

Congestive heart failure refers to a condition in which the heart cannot pump enough blood to the body's other organs. This condition can result from coronary artery disease, scar tissue on the heart cause by myocardial infarction, high blood pressure, heart valve disease, heart defects, and heart valve infection. Treatment programs typically consist of rest, proper diet, modified daily activities, and drugs such as angiotensin-converting enzyme (ACE) inhibitors, beta blockers, digitalis, diuretics, vasodilators. However, the treatment program will not reverse the damage or condition of the heart.

Hepatitis C is an infectious disease in the liver, caused by hepatitis C virus. Hepatitis C can progress to scarring (fibrosis) and advanced scarring (cirrhosis). Cirrhosis can lead to liver failure and other complications such as liver cancer. Current treatments include use of a combination of pegylated interferon alpha and the antiviral drug ribavirin. Success rates can vary between 50-80% depending on the virus genotype.

Head trauma refers to an injury of the head that may or may not cause injury to the brain. Common causes of head trauma include traffic accidents, home and occupational accidents, falls, and assaults. Various types of problems may result from head trauma, including skull fracture, lacerations of the scalp, subdural hematoma (bleeding below the dura mater), epidural hematoma (bleeding between the dura mater and the skull), cerebral contusion (brain bruise), concussion (temporary loss of function due to trauma), coma, or even death. Treatment for head trauma will vary with the type of injury. If the brain is damaged, there is no quick means to fix it, and often the damage may be irreversible by available treatment means.

Lung disease is a broad term for diseases of the respiratory system, which includes the lung, pleural cavity, bronchial tubes, trachea, upper respiratory tract, and nerves and muscles for breathing. Examples of lung diseases include obstructive lung diseases, in which the bronchial tubes become narrowed; restrictive or fibrotic lung diseases, in which the lung loses compliance and causes incomplete lung expansion and increased lung stiffness; respiratory tract infections, which can be caused by the common cold or pneumonia; respiratory tumors, such as those caused by cancer; pleural cavity diseases; and pulmonary vascular diseases, which affect pulmonary circulation. Treatment for lung disease varies according to the type of disease, but can include medication such as corticosteroids and antibiotics, oxygen, mechanical ventilation, radiotherapy, and surgery.

Depression is a mental disorder characterized by a low mood accompanied by low self-esteem and loss of interest or pleasure in normally enjoyable activities. Biologically, depression is accompanied by altered activity in multiple parts of the brain, including raphe nuclei, which are a group of small nuclei in the upper brain stem that is a source of serotonin; suprachiasmatic nucleus, which controls biological rhythms such as the sleep/wake cycle; hypothalamic-pituitary-adrenal axis, which is a chain of structures that are activated during the body's response to various stressors; ventral tegmental area, which is considered to be responsible for the “reward” circuitry of the brain; nucleus accumbens, which us thought to play a role in reward, laughter, pleasure, addiction, and fear; and the anterior cingulated cortex, which is activated by negative experiences. Treatments for depression include antidepressants that increase the amount of extracellular serotonin in the brain, exercise, and psychotherapy. However, the efficacy of these treatments continues to be questioned.

Non-obstructive azoospermia is a medical condition of a male not having any measureable level of sperm in his semen due to a problem with spermatogenesis. This is often caused by hormonal imbalance and can be treated using medications which restore the imbalance.

Andropause is a menopause-like condition experienced in middle-aged men, which involves a reduction in the production of the hormones testosterone and dehydroepiandrosterone. Treatments include hormone replacement therapy and exercise.

Scleroderma is a chronic autoimmune disease that affects connective tissue. Hardening of the skin is the most visible manifestation of the disease, although it can affect connective tissue throughout the body. There is no known direct cure for scleroderma.

Psoriasis is a chronic autoimmune disease that causes red, scaly patches to appear on the skin. The cause is psoriasis is linked to the excessive growth of skin cells. One hypothesis suggests that it is linked to T-cells which migrate to the dermis and trigger the release of cytokines that induce the rapid production of skin cells. Treatments for psoriasis include drugs that target the T-cells.

Retinitis pigmentosa is type of progressive retinal dystrophy in which the photoreceptors or the retinal pigment epithelium is abnormal and leads to visual loss. Therapies for treating retinitis pigmentosa are limited

The conditions described herein may be treated with a particular type, or a combination of types, of target cells. In preferred embodiments, the condition described herein may be treated by infusion of the cell types outlined in Table 2.

TABLE 2
Treatment regimes of various conditions using reprogrammed, i.e.,
retrodifferentiated or transdifferentiated or redifferentiated, target cells

| | Treatment Cell Type |
|---|---|
| Condition | (single or a combination thereof) |
| Aplastic Anemia | haematopoietic cells |
| Beta-thalassemia | haematopoietic cells |
| Diabetes | mesenchymal stem cells, pluripotent stem cells and/ |
| | or islet cells |
| Motor Neuron Disease | pluripotent stem cells, alveolar epithelium cells, |
| | ectoderm cells and/or neurons |
| Parkinson's Disease | pluripotent stem cells and/or neurons |
| Spinal Cord Injury | pluripotent stem cells and/or neurons |
| Muscular Dystrophy | pluripotent stem cells, mesenchymal stem cells |
| | and/or skeletal muscle cells |
| Kidney Disease | Kidney cells, mesenchymal stem cells and/or |
| | pluripotent stem cells |
| Multiple Sclerosis | pluripotent stem cells, mesenchymal stem cells |
| | and/or neurons |
| Congestive Heart Failure | cardiomyocytes, mesenchymal stem cells, |
| | pluripotent stem cells and/or endothelium cells |
| Hepatitis C Virus | haematopoietic cells, pluripotent stem cells, |
| | mesenchymal stem cells, and/or hepatocytes of |
| | the liver |
| Human Immunodeficiency Virus | haematopoietic cells |
| Head Trauma | pluripotent stem cells and/or neurons |
| Lung Disease | pluripotent stem cells, mesenchymal stem cells, |
| | alveolar epithelium cells and/or endothelium cells |
| Depression | pluripotent stem cells and/or neurons |
| Non-obstructive Azoospermia or | pluripotent stem cells, pluripotent germ cell, |
| Andropause | and/or sperm |
| Menopause and infertility | pluripotent stem cells, oocytes, pluripotent germ |
| | cells |
| Rejuvenation | pluripotent germ cells, pluripotent stem cells, |
| | keratinocytes, dermal papilla cells, neurons, |
| | sperm, osteocytes, chondrocytes, cardiomyocytes, |
| | skeletal muscular cells, neurons, endothelium |
| | cells, and/or melanocytes |
| Scleroderma Ulcers | pluripotent stem cells, endothelium cells, |
| | keratinocytes and/or mesenchymal stem cells |
| Psoriasis | mesenchymal stem cells and or pluripotent stem |
| | cells |
| Wrinkles | mesenchymal stem cells, keratinocytes, and/or |
| | pluripotent stem cells |
| Liver cirrhosis | hepatocytes of the liver, mesenchymal stem cells, |
| | endoderm cells, pluripotent stem cells |
| Autoimmune Disease | mesenchymal stem cells and/or pluripotent stem |
| | cells |
| Alopecia | dermal papilla cells and/or melanocytes |
| Retinitis pigmentosa or crystalline | neurons and/or pluripotent stem cells |
| dystrophy/blindness | |

By way an example, a patient may be treated for a condition as described above through the following steps:

1) Fistula-cannula is inserted in a patient's arm;

2) White blood cells are harvested through aphaeresis using an automated system, such as the COBE® Spectra Device (Gambro PCT);

3) Autologous retrodifferentiated stem cells are produced from the patient's white blood cells;

4) The autologous retrodifferentiated stem cells are washed and then infused intravenously into the patient;

5) Patient's progress is monitored, including taking blood tests and assessing the injured areas.

The invention will now be further described by way of the following non-limiting examples which further illustrate the invention, and are not intended, nor should they be interpreted to, limit the scope of the invention.

### EXAMPLES

### Example 1

### Materials and Methods

This clinical study assessed the safety of infusing a single dose of autologous 3 hr reprogrammed cells following exposure to haematopoietic inductive culture condition into four patients with aplastic anemia.

This clinical study was approved by the ethical committee of the King Edward Memorial (KEM) Hospital and was performed in joint collaboration with the Institute of Immunohematology (IU). Patients were required to fulfill the criteria outlined in Table 3. As a result, four patients with severe (3 males) and hypo-plastic (1 female) anaemia were enrolled into the study. These 4 patients were selected and monitored by IIH/KEM staff.

The patients' clinical and treatment history are described in Table 4, while their CD34⁺ cells infusion dosage are shown in Table 5.

TABLE 3
Inclusion Criteria

| | |
|---|---|
| Each | 1 - Absolute Neutrophil count <0.5 × 10⁹/L |
| criterion | 2 - Platelet count <20 × 10⁹/L |
| is | 3 - Anemia with corrected Reticulocyte <1% |
| Required | |
| Only One | 4 - Bone marrow cellularity <25% |
| of the | 5 - Bone marrow cellularity <50% with fewer than 30% |
| Criteria is | hematopoietic cells |
| Required | 6 - Subject evaluated within first 3 months of diagnosis |
| | 7 - Subject did not receive prior immunosuppressive therapy |

The patients were transfused with 2 units of irradiated packed red blood cells and 4 units of platelets to maintain their hemoglobin level above 8 g/dl and platelets counts above 50,000. Patient were apheresed by processing 2-3 times their total blood volume using the Cobe Spectra apheresis machine and the white blood cells separation kit (both from Gambro BCT). Apheresis involved jugular and anticubetal venous catheterization with single lumen catheter for venous access.

An aliquot of cells were collect aseptically for CD34 analysis following collection of 150-200 ml of buffy coat. Thereafter, the buffy coat was subjected to reprogramming under hematopoietic inductive culture condition. Briefly, the reprogramming procedure involved the addition of 1000 μg of purified CR3/43 (specially prepared by DakoCytomation for

TriStem Corp.) diluted in 30 ml of Iscove modified media, aseptically into the white blood cell bag. The bag was then incubated in a sterile tissue culture incubator maintained at 37° C. and 5% CO₂ for three hours. Following completion of the reprogramming process, the converted cells were analyzed for CD34⁺ cell content. Thereafter, cells were washed twice with saline solution using the cobe cell processor 2991. Upon agitation and re-suspension in saline solution, the cell suspension was infused into the patient via the jugular vein under gravity using an infusion set. Vital signs including CBC counts of patients were continuously monitored before and following infusion of the autologous reprogrammed cells.

TABLE 4
Clinical and treatment history of aplastic anemia patients up to Autologous human
reprogrammed stem cell (HRSC) infusion.

| Patient | Clinical History Up to HRSC infusion | Treatment up to RSC infusion |
|---|---|---|
| Patient A | Diagnosed with SAA in 2002 | Received a trial of anabolic |
| 25 yrs male | Presented with symptoms of weakness | steroid (TabMenabol) for 8 |
| Severe Aplastic | and dysponoea | months without any |
| Anaemia | Frequent episodes of rectal and gum | significant improvement |
| | bleeding and vomiting | |
| | Receives 4 and 2 units of blood and | |
| | platelets respectively, every month | |
| | Appear Jaundiced with severe eye | |
| | congestion | |
| Patient B | Diagnosed with Hypoplastic anaemia in | Received Haematics since 3 |
| 26 yrs female | January 2004 | months prior infusion of |
| Hypoplastic | Presented with symptoms of | RSC with no response |
| anemia | polymenorrhagia of 6 months duration | |
| | Only prior RSC infusion patients | |
| | received 4 and 6 units, respectively, of | |
| | packed red blood cells and platelets, | |
| | respectively | |
| Patient C | Diagnosed with very severe aplastic | Received cyclosporine |
| 19 yrs male | anaemia in 2003 | therapy for 6 months which |
| Very severe | Presented with anaemia, weakness, | ended in march 2003 with no |
| aplastic anaemia | dysponea on exertion | effect |
| | Fever with chills lasting 10-15 days due | |
| | to severe neutropaenia | |
| | Multiple episodes of infections, | |
| | vomiting and purpuric spots | |
| | Gluteal abscess | |
| | Fainted twice due to brain hemorrhage | |
| | Receives 5 and 2 units of blood and | |
| | platelets respectively every 2 weeks | |
| Patient D | Anaemia, weakness, dysponea | Received cyclosporine and |
| 35 yrs male | Fever with chills due to severe | anti-tuberculosis therapies. |
| Very severe | neutropenia and multiple episodes of | Did not respond to 6 months |
| Aplastic | infections and bleeding | of immunosuppression. |
| anaemia | Diagnosed 3 years ago | |
| | Receives 4 and 2 units of blood and | |
| | platelets, respectively, every month | |

All clinical monitoring was carried out by the IIH/KEM staff. Transfusion requirement was determined prior and post infusion from transfusion records obtained following receiving any unit of blood product. The transfusion units are only utilized following obtaining consent from patients and immediate relatives as witness. Prior transfusing the patient all blood products were irradiated at the Tata Memorial Hospital. Patients were also asked questions regarding their well-being prior and post infusion of the reprogrammed cells. All patients carried a copy or original records of their conditions and all laboratory and clinical follow up. The duration of monitoring of these patients was originally set for 2 years, but was extended. For the first month post infusion, patients were kept in hospital in sterilized positive pressure rooms.

TABLE 5
Patient, date of infusion, weight and height, mononuclear cells
collect and CD34+ cell infused.

| Patient ID and | | | Mononuclear | CD34⁺ |
|---|---|---|---|---|
| date of infusion | Weight | Height | cell collected | Received/kg |
| Patient A | 48 | 152 | 1 × 10⁹ | 11.7 × 10⁶ |
| Jul. 6, 2004 | | | | |
| Patient B | 38 | 153 | 3.6 × 10⁹ | 20 × 10⁶ |
| Jul. 14, 2004 | | | | |
| Patient C | 52 | 166 | 3.9 × 10⁹ | 25 × 10⁶ |
| Jul. 27, 2004 | | | | |
| Patient D | 52.5 | 160 | 4.3 × 10 | 23 × 10⁶ |
| Aug. 17, 2004 | | | | |

One million cells were stained according to the manufacturer instructions with the following panels of monoclonal antibodies (all from DakoCytomation):

Panel 1 consisted of Isotype negative control IgG1-FITC, IgG1-PE-Cy5 and IgG1-RPE conjugates

Panel 2 consisted of anti-human CD45-FITC and CD34-RPE-Cy5

Panel 3 consisted of anti-human CD38-FITC and CD34-RPE-Cy5

Panel 4 consisted of CD61-FITC and CD34-RPE-Cy5

Panel 5 consisted of CD33/13 RPE and CD7-FITC

Panel 6 consisted of CD45 and Glycophorin-A-RPE

Panel 7 consisted of CD3-FITC and CD19-RPE

Cell analysis was performed with a FACSCalibur system (BD bioscience) using the BD cell Quest software.

For clonal assay, bone marrow mononuclear cells (MNC) of patient prior and post infusion of the reprogrammed cells were seeded into methocult GFH4434 supplemented with recombinant growth factors according to the manufacturer's instructions (Stem Cell Technologies). Differentiation into haematopoietic cell colonies was assessed and scored with time using phase contrast inverted microscopy.

Patient CBC, liver enzymes and haemoglobin variants were continuously monitored before and post procedure. Following release from hospital patients, CBC, liver enzymes, hemoglobin variants, and peripheral blood karyotyping and G banding were monitored by an independent laboratory for reconfirmation purposes. These tests were performed frequently following infusion of the autologous reprogrammed cells.

Peripheral blood samples and bone marrow cells were analyzed before and following infusion of the autologous reprogrammed cells. This test was repeated in six-month intervals for the first year and on a yearly basis following 2 years post-initiation of autologous reprogrammed stem cell therapy. In addition, reprogrammed cells were analyzed prior infusion to look into the stability of the cells, which was also performed following the 3 hr conversion step, as well as post-establishment of a maximum 1 month long term culture of the converted cells. Karyotyping and G banding were monitored by a third independent laboratory.

Bone marrow smears and trephine section was performed before and post infusion of the autologous reprogrammed cells. This test was performed 14-20 days post infusion of the autologous reprogrammed cells and thereafter on a yearly basis.

All smear and trephine sections were scanned using a microscope hooked to a camcorder before and following infusion of the reprogrammed stem cells to assess and keep record of engraftment.

### Results

All patients tolerated the aphaeresis and the single reprogrammed stem cells infusion procedure with no adverse event. Patient A and Patient D became transfusion-independent post single infusion of reprogrammed haematopoietic stem cells (RHSC) (see Tables 6 and 7). Platelets, neutrophil and red blood cell engraftment ensued 3 and 6 days post infusion in Patient A and Patient D, respectively. Fetal hemoglobin switching was noted in Patient A and Patient D (see Tables 4 and 5) but not in Patient B and Patient C (data not shown). Prior to infusion, liver enzymes were elevated in Patient A and Patient D despite being negative for HCV (as measured by ELISA). Liver enzymes started to normalize post infusion of RHSC and reached normal levels 4 years post infusion of RHSC. Patient B and Patient C died 2 years and six months post infusion, respectively. Fetal Hb switching was noted in patients 001 and 004 (see Tables 6 and 7). These two patients exhibited long term engraftment post single infusion of the autologous HRSC.

TABLE 6
Patient A's complete blood counts, hemoglobin variant and liver enzymes of a
severe aplastic anemia patient before and after in fusion of autologous HRSC.

| | Nadir prior | Jul. 19, 2004 | May 26, 2005 | Mar. 07, 2006 | Jan. 05, 2008 |
|---|---|---|---|---|---|
| Blood test | to infusion | Post-infusion | Post-infusion | Post-infusion | Post-infusion |
| WBC [10³/μL] | 1.3 | 3.4 | 3.1 | 3.7 | 5.4 |
| HB [g/dL] | 2.6 | 7.1 | 9 | 12 | 14 |
| RBC [10⁶/μL] | 0.9 | 2 | 2.3 | 2.9 | 3.86 |
| RETIC [%] | 1 | | 4 | | 1.7 |
| MCV [fL] | 101.5 | 101.5 | 121.74 | 100 | 101.4 |
| MCH [Pg] | 33.6 | 35.5 | 39.13 | 30.8 | 36.3 |
| MCHC [g/dL] | 33.8 | 35 | 32.14 | 30.8 | 35.8 |
| PLATELETS | 18 | 26 | 37 | 58 | 189 |
| [10³/μL] | | | | | |
| NEUTROPHILS | 0.32 | 1.000 | 1.054 | 1.184 | 1.994 |
| [10³/μL] | | | | | |
| ESO [10³/μL] | | | 0.62 | 0.74 | 0.65 |
| BASO [10³/μL] | | | 0 | 0 | 0.054 |
| LYMPH [10³/μL] | | 1.500 | 1.922 | 2.220 | 2.214 |
| MONO [10³/μL] | | 0.500 | 0.062 | 0.222 | 0.324 |
| HB A [g/dL] | | 5.1 | 6.7 | 9.2 | 12 |
| HBA2 [g/dl] | | 0.13 | 0.14 | 0.24 | 0.39 |
| HBF [g/dl] | ND | 1.2 | 2.4 | 2.6 | 1.82 |
| SGOT [U/L] | 150 | 127 | 74 | 32 | 30 |
| SGPT [U/L] | 145 | 181 | 49 | 37 | 44 |

WBC = white blood cell;
HB = hemoglobin;
RBC = red blood cell;
RETIC = reticulocyte;
MCV = mean corpuscular volume;
MCH = mean corpuscular hemoglobin;
MCHC = mean corpuscular hemoglobin concentration;
ESO = eosinophils;
BASO = basophils;
LYMPH = lymphocytes;
MONO = monocytes;
HB A = hemoglobin A;
HBA2 = hemoglobin A2;
HBF = fetal hemoglobin;
SGOT = serum glutamic oxaloacetic transaminase;
SGPT = serum glutamic pyruvic transaminase

TABLE 7
Patient D's complete blood counts, hemoglobin variant and liver enzymes of a
severe aplastic anemia patient before and after in fusion of autologous HRSC

| | Nadir prior | Jul. 19, 2004 | May 26, 2005 | Mar. 07, 2006 | Jan. 05, 2008 |
|---|---|---|---|---|---|
| Blood test | to infusion | Post-infusion | Post-infusion | Post-infusion | Post-infusion |
| WBC [10³/μL] | 1.3 | 3.4 | 3.1 | 3.7 | 5.4 |
| HB [g/dL] | 2.6 | 7.1 | 9 | 12 | 14 |
| RBC [10⁶/μL] | 0.9 | 2 | 2.3 | 2.9 | 3.86 |
| RETIC [%] | 1 | | 4 | | 1.7 |
| MCV [fL] | 101.5 | 101.5 | 121.74 | 100 | 101.4 |
| MCH [Pg] | 33.6 | 35.5 | 39.13 | 30.8 | 36.3 |
| MCHC [g/dL] | 33.8 | 35 | 32.14 | 30.8 | 35.8 |
| PLATELETS | 18 | 26 | 37 | 58 | 189 |
| [10³/μL] | | | | | |
| NEUTROPHILS | 0.32 | 1.000 | 1.054 | 1.184 | 1.994 |
| [10³/μL] | | | | | |
| ESO [10³/μL] | | | 0.62 | 0.74 | 0.65 |
| BASO [10³/μL] | | | 0 | 0 | 0.054 |
| LYMPH [10³/μL] | | 1.500 | 1.922 | 2.220 | 2.214 |
| MONO [10³/μL] | | 0.500 | 0.062 | 0.222 | 0.324 |
| HB A [g/dL] | | 5.1 | 6.7 | 9.2 | 12 |
| HBA2 [g/dl] | | 0.13 | 0.14 | 0.24 | 0.39 |
| HBF [g/dl] | ND | 1.2 | 2.4 | 2.6 | 1.82 |
| SGOT [U/L] | 150 | 127 | 74 | 32 | 30 |
| SGPT [U/L] | 145 | 181 | 49 | 37 | 44 |

WBC = white blood cell;
HB = hemoglobin;
RBC = red blood cell;
RETIC = reticulocyte;
MCV = mean corpuscular volume;
MCH = mean corpuscular hemoglobin;
MCHC = mean corpuscular hemoglobin concentration;
ESO = eosinophils;
BASO = basophils;
LYMPH = lymphocytes;
MONO = monocytes;
HB A = hemoglobin A;
HBA2 = hemoglobin A2;
HBF = fetal hemoglobin;
SGOT = serum glutamic oxaloacetic transaminase;
SGPT = serum glutamic pyruvic transaminase

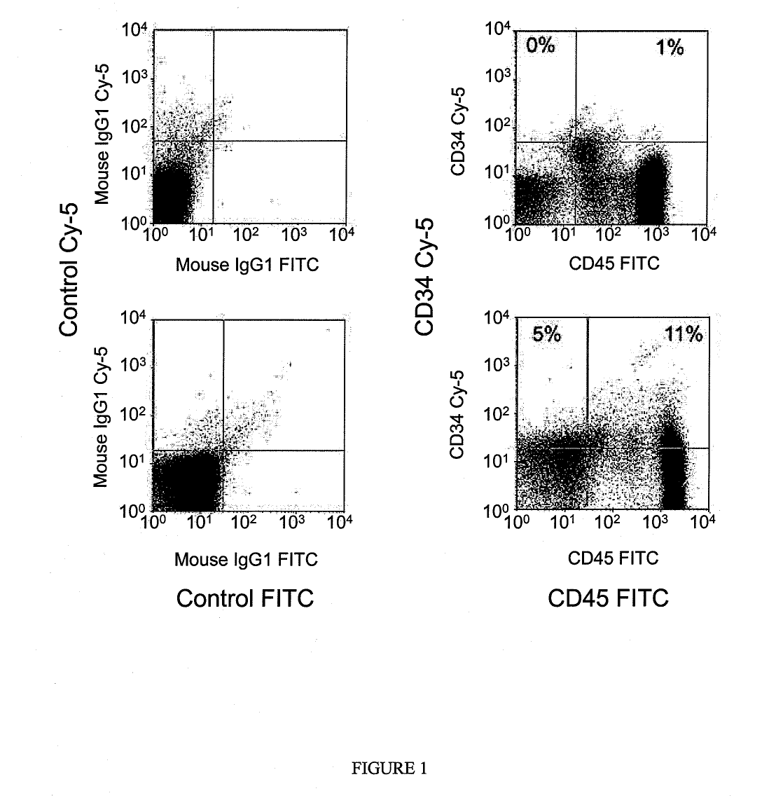

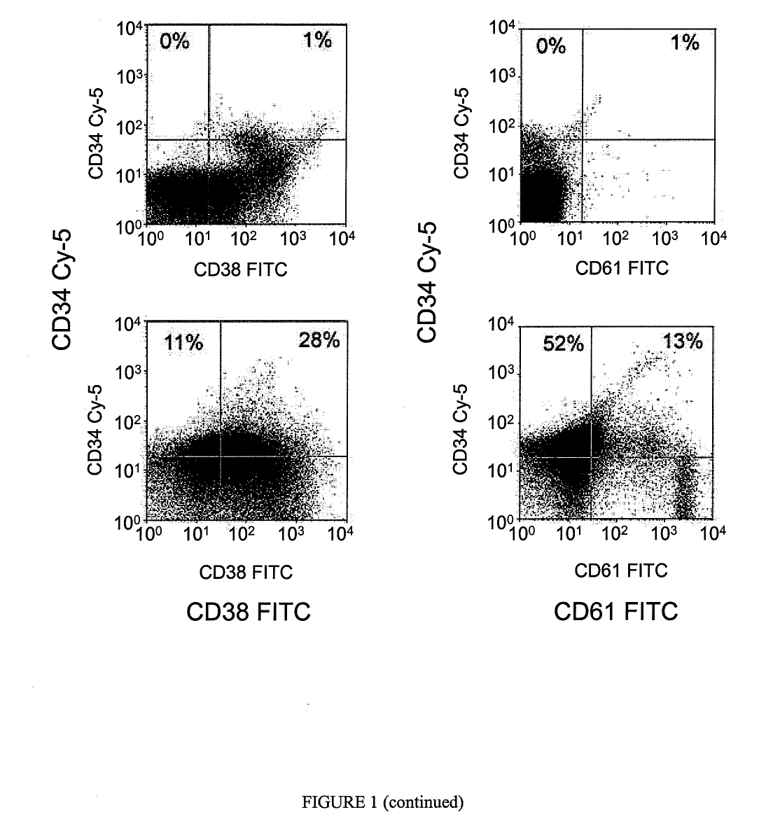

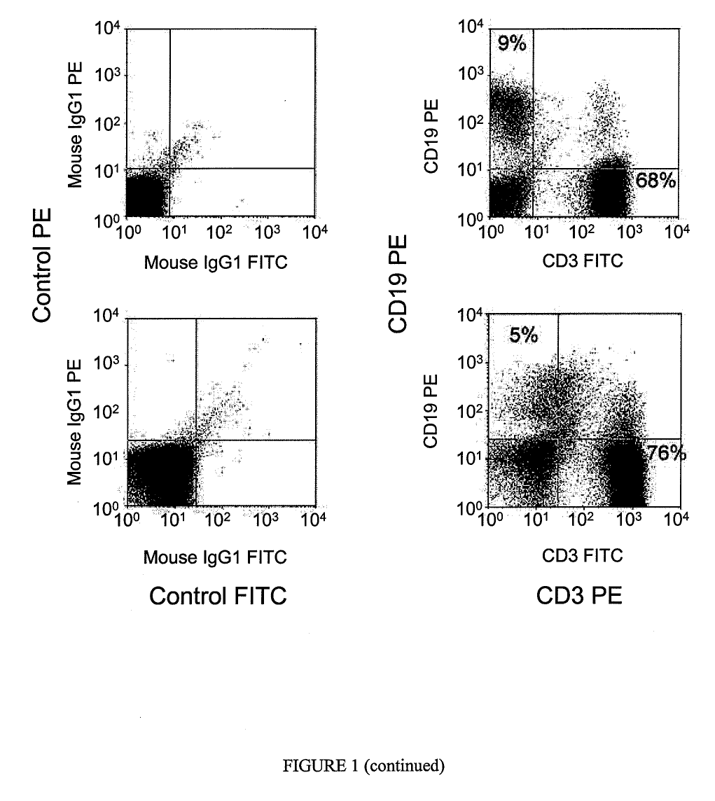

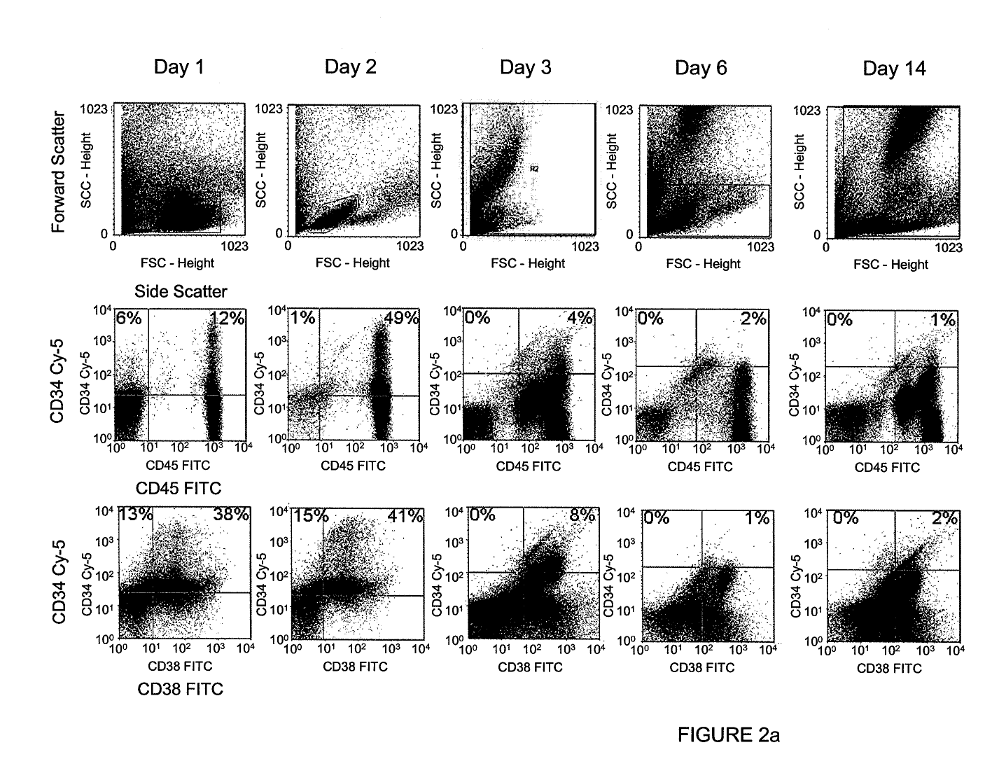

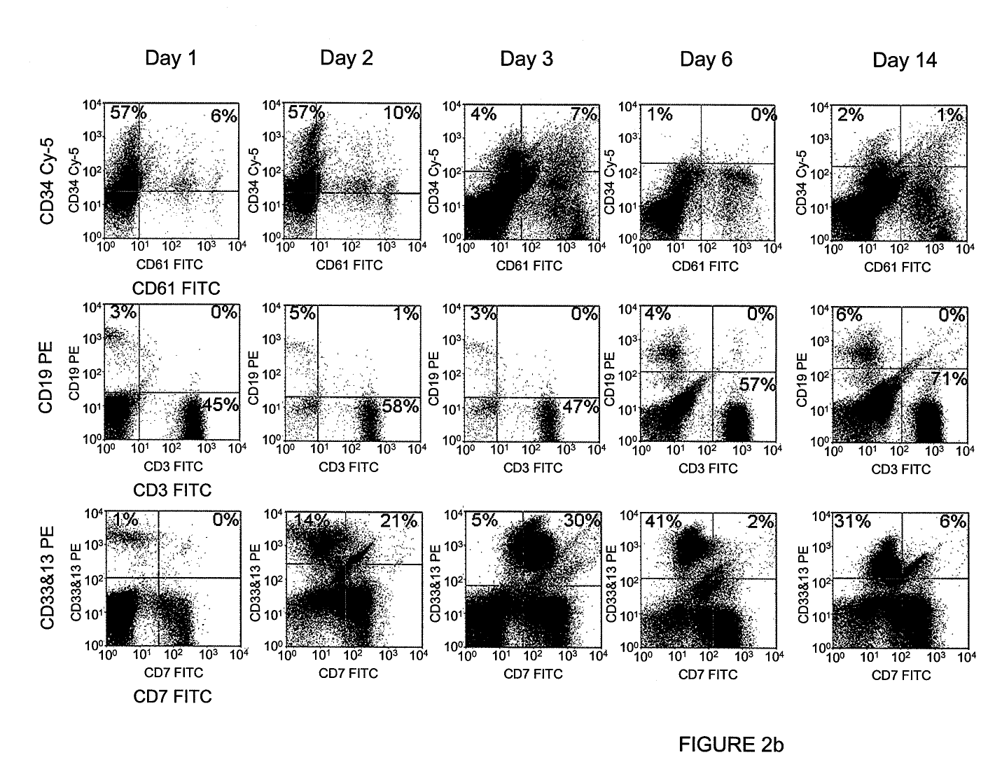

Flow cytometry of aphaeresed mononuclear cells before and 3 hrs post induction of haematopoietic reprogramming are shown in FIG. 1. The number of CD34 positive cell generated post haematopoietic reprogramming is listed in Table 4. A representative flow cytometry of apheresed mononuclear before and post haematopoietic reprogramming show significant increase in the number of CD34 positive cells with and without expression of CD45, CD38 and CD7 (FIG. 1). On infusion, the CD34 cells circulated peripheral blood for 3-6 days and thereafter differentiated at a sustainable level into myelocyte as depicted by significant increase in cells expressing CD33&13 with and without CD7 having high forward and side scatter (see FIG. 2). This pattern of reprogramming was observed in all patients.

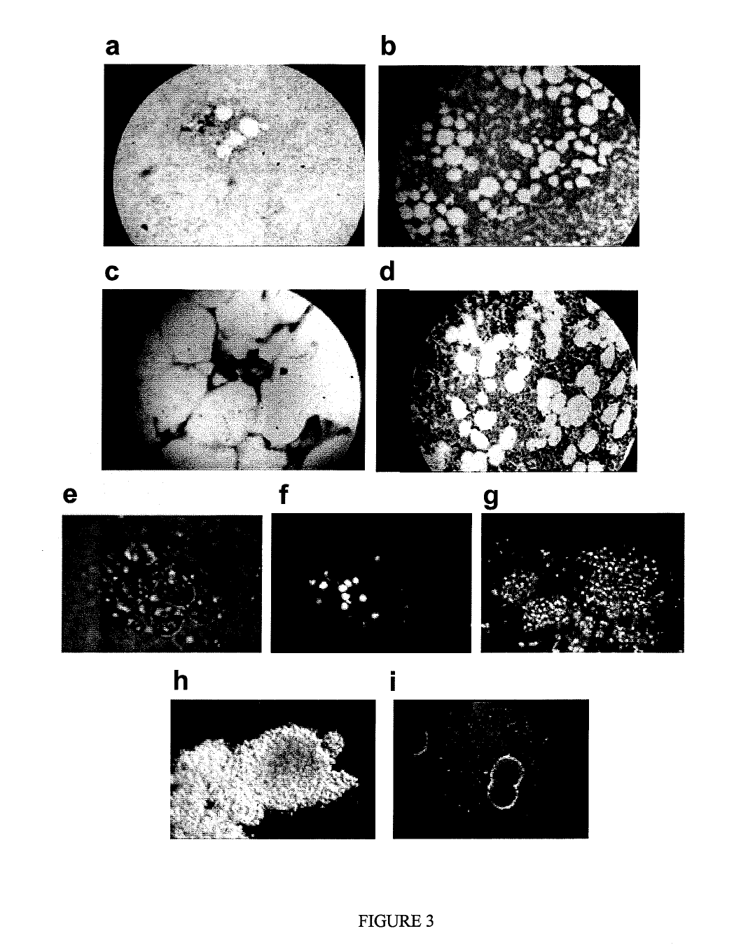

Hardly any colonies formed from patients bone marrow aspirate before infusion of the autologous HRSC following seeding into methylcellulose cell culture (see FIG. 3). Clonal assay only in Patient B was suffering from hypoplastic anaemia showed depressed haempoiesis. However, 14 days to 20 days post infusion all bone marrow aspirates obtained from patients gave rise to normal range of a variety of haematopoietic colonies with mild elevation in the number of Burst forming unit—erythroid (BFU-erythroid). Bone marrow smears and trephine sections (see FIG. 3) 14-20 days post infusion of autologous of HRSC showed significant increase in the number of myelocytes at various stages of differentiation with mature and immature megakaryocytes in all patients when compared to baseline. Erythroid hyperplasia at various stages of differentiation was also noted in all samples.

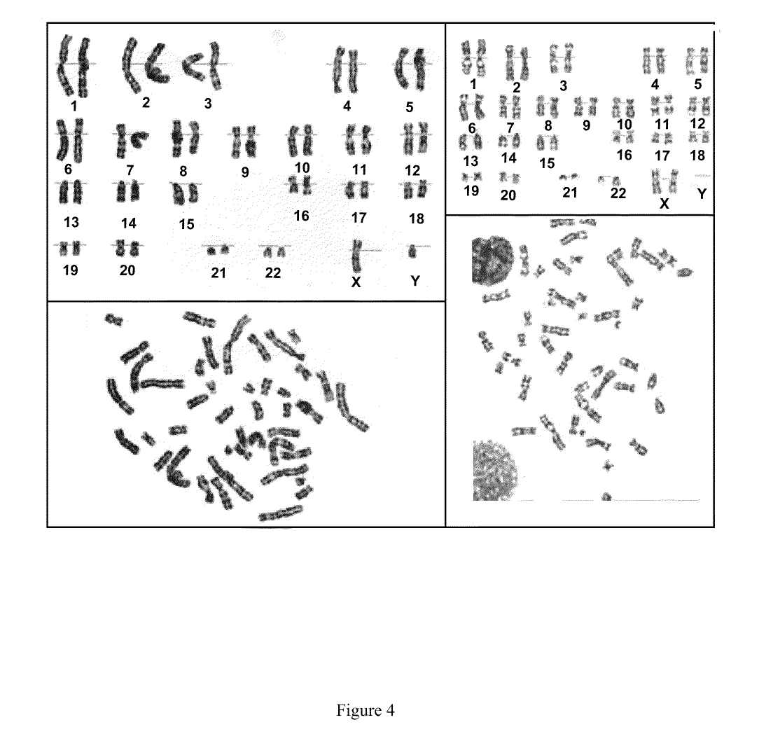

No changes in Karyotyping and G banding pattern in peripheral blood or bone marrow samples obtained before and after infusion (in Patient A and Patient D for up to more than 4 years) of autologous HRSC in all patients (see FIG. 4).

Post infusion of the autologous HRSC into aplastic anaemia patients primitive (CD38 negative) and committed (CD38 Positive) CD34 cells circulated the peripheral circulation for three days prior reprogramming into myelocytes (FIG. 2). Myeloid engraftment ensued 3 days post infusion of the HRSC in Patient A, Patient B, and Patient D. On the other hand myeloid engraftment ensued at day 20 for patient 003 when analyzed by flow cytometry. The single infusion of HRSC, without the use of any pre-conditioning regimen lead to long term engraftment of 2 out of 4 patients with aplastic anaemia. Patient A and Patient B showed long term engraftment without any transfusion post infusion of the HRSC. Engraftment of neutrophils, red blood cells and platelets in such patient was accompanied by switching or increase in Hb F haemoglobin level (Tables 6 and 7). This was not noted in the other 2 patients that died. Hb F switching in these two patients confirms the reprogramming capabilities of the infused HRSC towards juvenile Hb phenotype and hence engraftment and reconstitution as observed with cord blood stem cells transplant (Elhasid et al., Leukemia 2000, 14: 931-934; Locatelli et al., Bone Marrow Transplant 1996, 18: 1095-101).

The long term engraftment with preservation of chromosome number and banding reflect clearly the safety of infusing the HRSC in a haematological condition where clonal evolution is not a rare event with conventional therapies

Importantly, the autologous HRSC were capable of long term engraftment and survival rate in a subset of severe aplastic anaemia patients without use of any immunosuppression regiment, just like those seen with syngenic stem cells.

In summary, fourteen days after infusion, the analysis of the bone marrow of the infused patients showed an increase in bone marrow cellularity, and myeloid, erythroid and megakaryocytic lineages at various stages of differentiation with a drop in fat cells and stromal cells, which are the predominant occupant of severe aplastic anaemia bone marrow. There was a significant increase in red blood cell mass in the bone marrow with a corresponding increase in haemoglobin level as well as reticulocytes count. There was also a steady increase in foetal haemoglobi—an important component in ameliorating sickle cell anaemia and beta thalassemia—and foetal haemoglobin-expressing red blood cells following infusion. Furthermore, there was significant improvement in red blood cell indices as determined by red blood cell size, haemoglobin content and concentration. The reprogrammed cells have exhibited a normal karyotype and genetic stability after infusion. Finally, engraftment and long term repopulation was observed in 3 more patient suffering from aplastic anemia.

### Example 2

### Materials and Methods

Autologous reprogrammed haematopoietic cells (target cells) were tested in 21 patients with beta thalassemia. Nineteen patients had beta-thalassemia major and 2 had thalassemia intermedia. One of the beta thalassemia intermedia patient was thalassemia/Hb E variant (common in patient of far eastern and Indian origin) and the other had Thalassemia/Sickle cell anaemia.

Patients were apheresed by processing 2-3 times their total blood volume. The autologous reprogrammed cells were generated through reprogramming white blood cells until the target cells were obtained, as indicated by their distinguishing characteristics as described above. The patients were administered the autologous reprogrammed cells via intravenous infusion into the jugular or veins in the arm or thigh.

### Results

There was no toxic or adverse side effects were observed following infusion of the reprogrammed cells in patients with beta thalassemia as measured by vital sign monitoring, echocardiogram, bone densities, liver and kidney enzymes including Karyotyping and G-banding when compared to baseline. There was a statistically significant mean reduction (50%) in blood transfusion requirement in beta thalassemia major patients now nearly 9 months following infusion of the reprogrammed cells when compared to base line. Two thalassemic patient who are beta thalassemia intermediate (one was thalassemia/HB E and the other Thalassaemia /Sickle Cell Anaemia) were transfusion-independent now nearly 9 months following infusion of the reprogrammed cells.

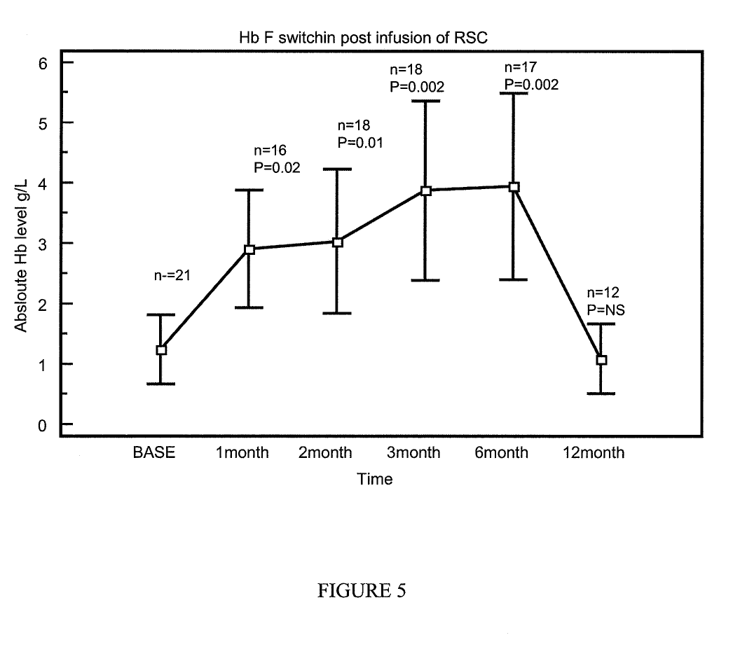

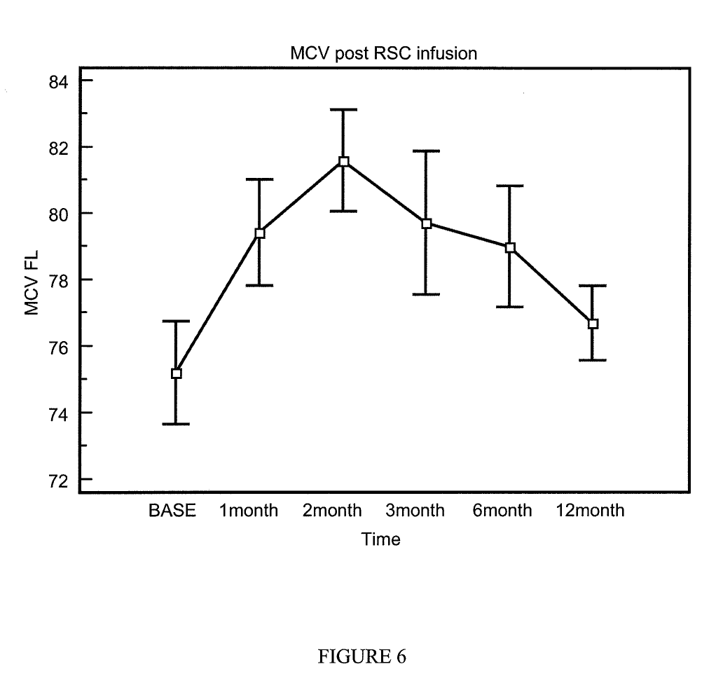

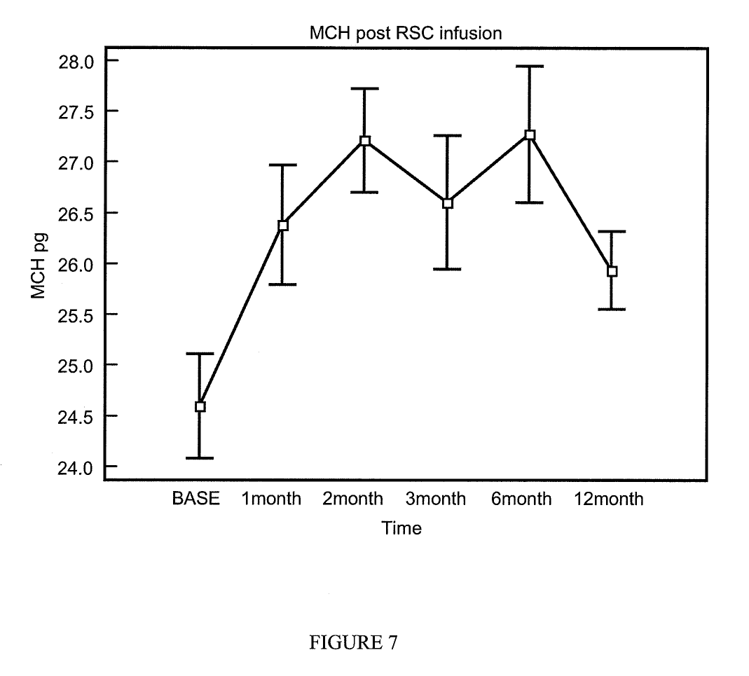

The mean weight and height following infusion of the reprogrammed cells were significantly greater when compared to base line, and the organ size in thalassemic patients with enlarged spleen and/or liver was normalized. The absolute mean fetal haemoglobin concentration significantly increased in patients with thalassemia major and intermedia following infusion of the reprogrammed cells when compared to baseline (FIG. 5). Also, the mean red blood cell indices as reflected by improvement in red blood cell size haemoglobin content (FIG. 6) and concentration (FIG. 7) was also significantly improved as compared to baseline.

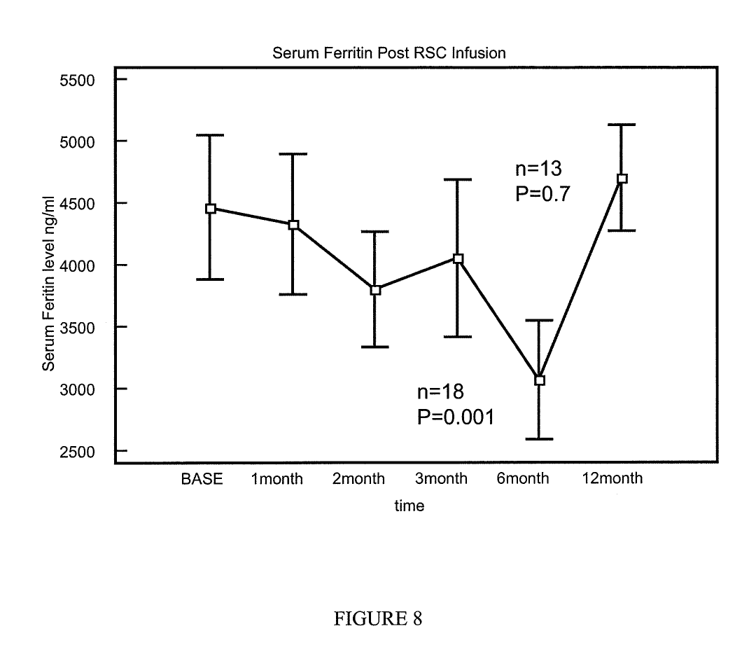

Finally, the mean serum ferritin (a biomarker for iron overload) significantly decreased in thalassemic patients following infusion of the reprogrammed cells (FIG. 8). Iron overload is the major cause of mortality and morbidity in patients with thalassemia; sickle cell anaemia and any transfusional iron overload induced disorder.

### Example 3

### Materials and Methods

Two patients with diabetes were apheresed by processing 2-3 times their total blood volume. Autologous reprogrammed mesenchymal stem cells, pluripotent stem cells, and islet cells (target cells) were obtained through reprogramming apheresed white blood cells until the target cells developed, as indicated by their distinguishing characteristics as described above. The patients were administered the autologous reprogrammed cells via intravenous infusion into the jugular or veins in the arm or thigh.

### Results

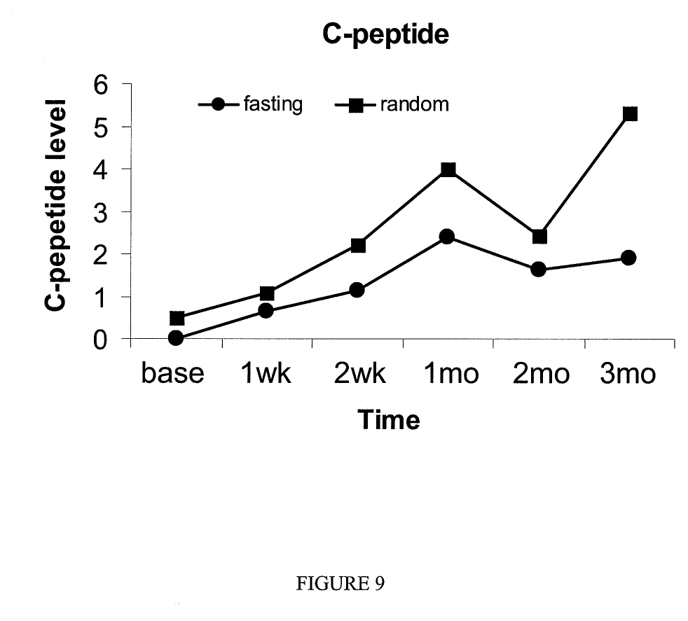

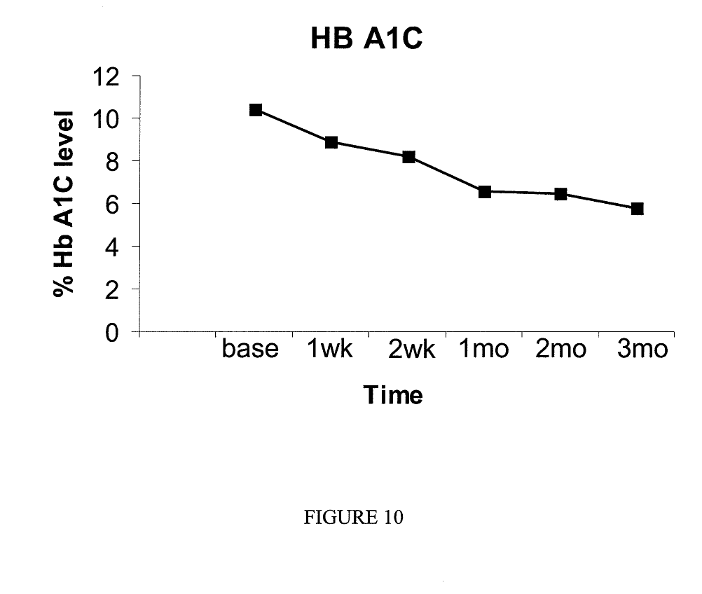

Following infusion of the autologous reprogrammed cells, the patients were synthesizing normal level of insulin, as measured by fasting and 90 minutes food intake stimulated c-peptide. This normal level of c-peptide is being maintained up to 3 months following infusion of the reprogrammed cells (FIG. 9). In addition, Hb A1C levels, which indicate glycemic control, had normalized following infusion of the reprogrammed cells (FIG. 10). For example patients with Hb A1C above 10% prior infusion have now Hb A1C of 5.8% following receiving the reprogrammed cells. Furthermore blood glucose levels of these patients appear to reach normal levels post infusion when compared to baseline. In addition a drastic reduction in insulin intake/injection by the diabetic patient was noted following infusion of the reprogrammed cell.

### Example 4

### Materials and Methods

Four patients with amyotrophic lateral sclerosis (ALS) received the autologous reprogrammed cells. The diagnosis of this disease does not involve a specific biomarker. This disease is diagnosed by clinical exclusion of other similar disorders.

Patients were apheresed by processing 2-3 times their total blood volume. Autologous reprogrammed pluripotent stem cells, alveolar epithelium cells, and neurons (target cells) were obtained through reprogramming apheresed white blood cells until the target cells developed, as indicated by their distinguishing characteristics as described above. The patients were administered the autologous reprogrammed cells via intravenous infusion into the jugular or veins in the arm or thigh.

### Results

In patients suffering from ALS and treated with the autologous reprogrammed cells, there was a significant improvement in Pulmonary Function test (PFT). Impairment in this lung function test is one of the causes that lead to early mortality. Most patients experienced less stiffness in limbs and neck, and some reported improvement in their speech. Others showed improvement in their walking ability as well as lifting of head.

### Example 5

### Materials and Methods

Four patients with Parkinson's disease were apheresed by processing 2-3 times their total blood volume. Autologous reprogrammed pluripotent stem cells and neurons (target cells) were obtained through reprogramming apheresed white blood cells until the target cells developed, as indicated by their distinguishing characteristics as described above. The patients were administered the autologous reprogrammed cells via intravenous infusion into the jugular or veins in the arm or thigh.

### Results

Patients in which the shiverer effect of the disease is pronounced experienced significant reduction in shaking and conventional medication intake used to manage this disorder. The first patient was on 4 tablets of Sinemet (dopamine regulator) daily and 4-months post-transfusion is only taking 1 tablet a day.

### Example 6

### Materials and Methods

Two patients with spinal cord injury were apheresed by processing 2-3 times their total blood volume. Autologous retrodifferentiated pluripotent stem cells and neurons (target cells) were obtained through reprogramming apheresed white blood cells until the target cells developed, as indicated by their distinguishing characteristics as described above. The patients were administered the autologous reprogrammed cells via intravenous infusion into the jugular or veins in the arm or thigh.

### Results

One patient was lost to follow up. The other patient was a quadriplegic with C5-C6 spinal cord injury. This patient was unable to sit up or rotate his torso in bed. Following treatment, he was able to sit upright for very long periods of time and able to turn his body in bed. He was also capable of standing alone following propping his body against a wall. Further, he was able to wiggle his toe and is reporting sensation in his bladder.

An MRI analysis before and following infusion of the reprogrammed cells showed slight reduction in lesion size following infusion of reprogrammed cells. He began actively undergoing physiotherapy and generally felt much better than before.

### Example 7

### Materials and Methods

Two patients with Muscular Dystrophy (MD) were treated with autologous reprogrammed pluripotent stem cells, mesenchymal stem cells, and skeletal muscle cells (target cells) were obtained through reprogramming apheresed white blood cells until the target cells developed, as indicated by their distinguishing characteristics as described above. The first patient was inflicted with limb-girdle MD, which refers to a class of MD wherein the muscles that are most severely affected generally those of the hips and shoulders, and the second patient was inflicted with nemelin MD.

Patients were apheresed by processing 2-3 times their total blood volume. The autologous reprogrammed cells were generated through reprogramming in accordance to the invention. The patients were administered the autologous reprogrammed cells via intravenous infusion into the jugular or veins in the arm or thigh.

### Results

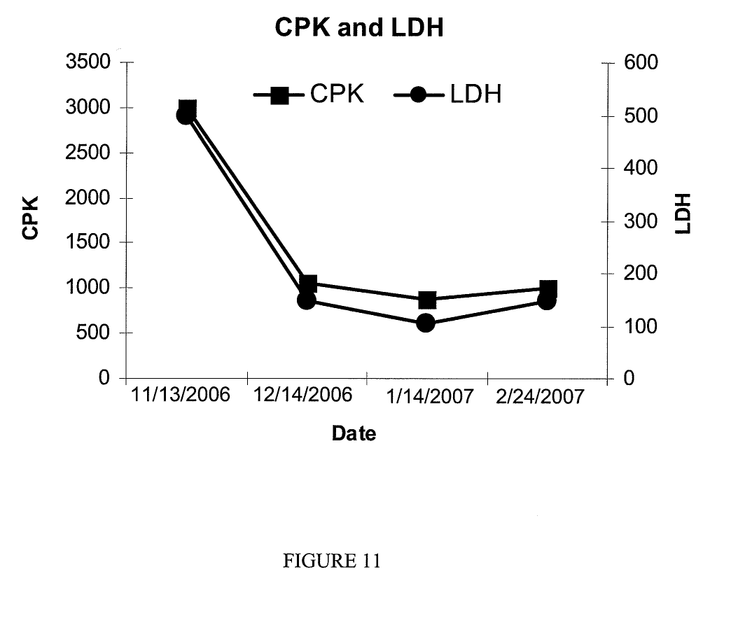

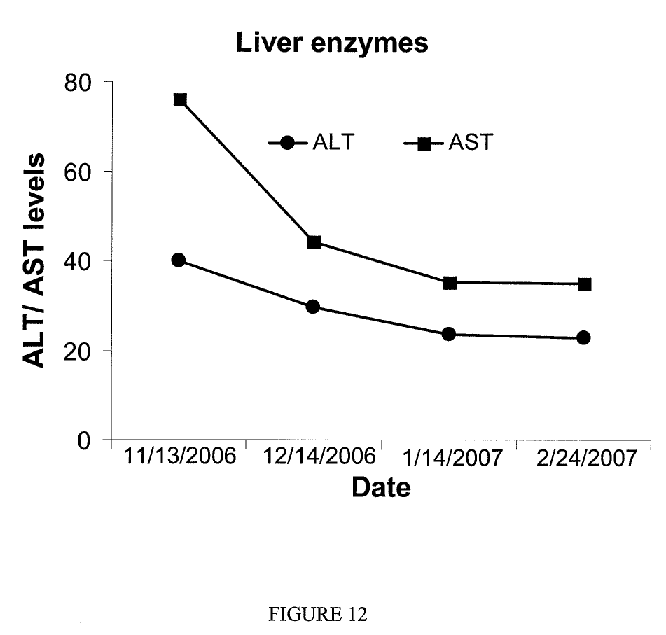

The muscle atrophy associated with MD can be measured by monitoring the level of the muscle enzyme creatine phosphokinase (CPK). This enzyme decreased in response to infusion of the retrodifferentiated stem cells (FIG. 11). Lactate dehydrogenase, an enzyme which is elevated during tissue breakdown, also decreased (FIG. 11). Patients also experienced a decrease in liver enzymes alanine aminotransferase (ALT) and aspartate aminotransferase (AST), which are both associated with inflammation and injury to liver cells, as well as skeletal muscle.

Moreover, patients showed an improvement in patient mobility as determined by cam recording of patients before and following infusion of the reprogrammed cells, and in pulmonary function test following infusion of the reprogrammed cells when compared to baseline

### Example 8

### Materials and Methods

Patients with kidney disease were treated with autologous reprogrammed pluripotent stem cells, mesenchymal stem cells, and kidney cells (target cells) were obtained through reprogramming apheresed white blood cells until the target cells developed, as indicated by their distinguishing characteristics as described above. The patients were apheresed by processing 2-3 times their total blood volume. The autologous reprogrammed cells were generated through reprogramming in accordance to the invention. The patients were administered the autologous reprogrammed cells via intravenous infusion into the jugular or veins in the arm or thigh.

### Results

Patients receiving an infusion of autologous reprogrammed cells experienced an improvement in various fluid marker levels which were indicative of healthier kidney function such as urine output, serum creatinine and BUN or UREA. For example, a 75-year old diabetic male patient showed improved kidney function 24 months after treatment with the autologous reprogrammed cells (see Table 8). The patient showed increased levels in hemoglobin, which is the protein molecule in red blood cells that carries oxygen, and insulin-like growth factor-1 (IGF-1), a growth factor. The patient also showed decreases in urea, which is an organic compound, creatinine, which is a breakdown product of creatine phosphate in muscle, uric acid, which is an organic compound excreted in urine, and phosphorus, which is a mineral found in bone; high amount of these markers are indicative of poor kidney function. Further, the patient showed an decrease in glycosylated hemoglobin (HbA1C), which is a form of hemoglobin and is commonly used as an indicator of plasma glucose concentration of people suffering from diabetes.

Similarly, a 51-year old diabetic male exhibited similar improvement 12 months after treatment (see Table 8). This patient showed increased levels of hemoglobin and decreased levels of creatinine, HbA1C, and blood urea nitrogen (BUN), which is a measurement of the amount of nitrogen in the blood in the form of urea.

TABLE 8
Kidney function marker levels in two diabetic male patients.

| | 75-Year | | 51-Year | |
|---|---|---|---|---|
| | Old Diabetic Male | | Old Diabetic Male | |
| | | 24-mos post- | | 12-mos post- |
| | Baseline | treatment | Baseline | treatment |
| Hemoglobin [g/dl] | 8 | 12.9 | 12 | 14.5 |
| Urea [g/dl] | 190 | 38 | n/a | n/a |
| Creatinine [mg/dl] | 12 | 1.9 | 15 | 4.5 |
| Uric acid [mg/dl] | 8.5 | 4 | n/a | n/a |
| Phosphorus [mg/dl] | 6.1 | 3.8 | n/a | n/a |
| HbA1C [%] | 14.4 | 6.1 | 12 | 6 |
| IGF-1 [ng/dl] | 45 | 112 | n/a | n/a |
| BUN [mg/dl] | n/a | n/a | 79 | 19 |

HbA1C = glycosylated hemoglobin;
IGF-1 = Insulin-like Growth Factor 1;
BUN = blood urea nitrogen

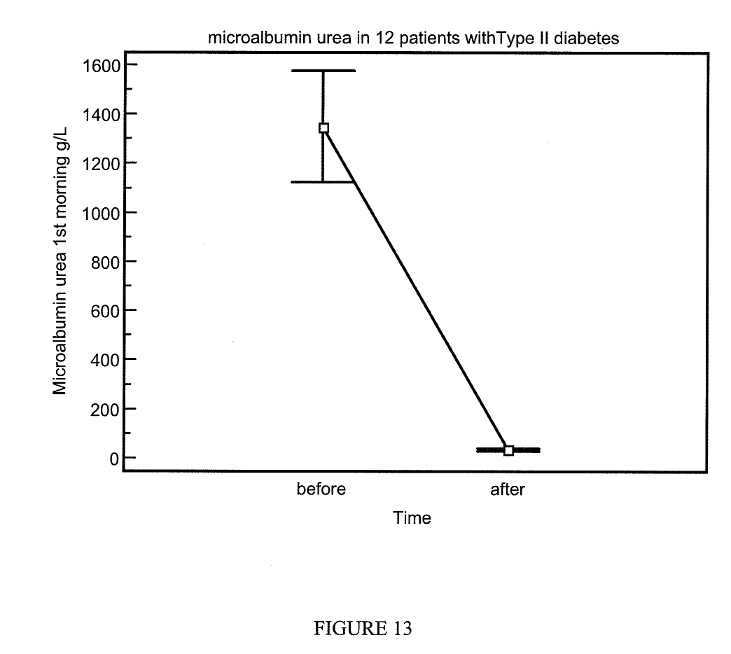

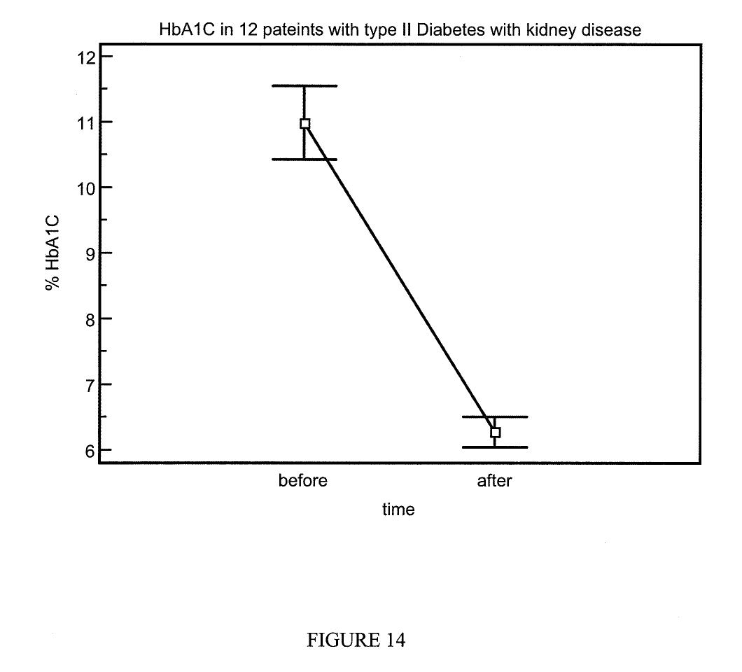

Analysis of 12 patients having Type II diabetes and treated with autologous reprogrammed cells revealed decreased levels of microalbumin (FIG. 13), which is associated with leakage of albumin into the urine and is an indicator of kidney disease, as well as vascular endothelial dysfunction and cardiovascular disease. The 12 patients also experienced decreased levels of HbA1C (FIG. 14).

In addition, a 45-year old female suffering from autoimmune glomerulonephritis exhibited a improvement in kidney function within 18-months after treatment with autologous reprogrammed cells (see Table 9).

TABLE 9
Kidney function marker levels in 45-year old female
patient suffering from autoimmune glomerulonephritis.

| | Baseline | 18-mos post-treatment |
|---|---|---|
| Hemoglobin [g/dl] | 9 | 11.5 |
| Creatinine [mg/dl] | 9.3 | 2.1 |
| BUN [mg/dl] | 89 | 15 |

BUN = blood urea nitrogen

Furthermore, a 59-year old female patient suffering from end stage renal disease showed improvement in creatinine level, urea, hemoglobin level, and parathyroid hormone (PTH) upon treatment with reprogrammed cells (see Table 10). Kidney function marker levels in 45-year old female patient suffering from autoimmune. The patient also decreased in the number of hemodialysis sessions that she attended per month, from 12 sessions to about 8 sessions.

TABLE 10
Kidney function marker levels in 59-year old female patient suffering from end
stage renal disease before and after therapy with reprogrammed cells.

| Kidney Function | Pre-therapy | Post-therapy | | | |
|---|---|---|---|---|---|
| Marker | Mar. 02, 2009 | Apr. 29, 2009 | May 23, 2009 | Aug. 05, 2009 | Aug. 11, 2009 |
| Hemoglobin [g/dl] | 9.8 | 9.2 | 11.5 | | |
| TLC/WBC (per μl) | 3800 | 6800 | 5100 | | |
| PLT (per μl) | 361000 | 273000 | 348000 | | |
| HbA1C [%] | 5.6 | | | | |
| AST [U/l] | 25 | 16 | 14 | | |
| ALT [U/l] | 49 | 11 | 12 | | |
| Urea [mg/dl] | 90 | 70 | | | 22.13 |
| Creatinine [mg/dl] | 14.5 | 11.2 | 11.86 | 8.86 | 9.58 |
| S. Albumin [g/dl] | 3 | 3.4 | 3.3 | | 3 |
| PT [seconds] | 31.2 | | | | |
| INR | 2.71 | | | | |
| Calcium [mg/dl] | 8.4 | 9.5 | 9.4 | 8.7 | 9.5 |
| Phosphorus [mg/dl] | 8 | 9.2 | 5.9 | | |
| Potassium | 5.9 | | 5.2 | 6.9 | 4.1 |
| [mmol/dl] | | | | | |
| Sodium [mmol/dl] | 133 | | | 136 | |
| Uric acid [mg/dl] | 7.9 | 6.1 | 7.5 | | 4.8 |
| PTH [pg/ml] | | | 195.6 | | 108.2 |

TLC = total leukocyte count;
WBC = white blood cells;
PLT = platelet;
HbA1C = glycosylated hemoglobin;
AST = aspartate aminotransferase;
ALT = alanine aminotransferase;
PT = pro-thrombin time;
INR = international normalized ratio (for blood coagulation);
PTH = parathyroid hormone

A 46-year old patient suffering from chronic renal failure due to diabetes also showed improvement in creatinine level, urea, and hemoglobin level upon treatment with reprogrammed cells (see Table 11). The patient decreased in the number of hemodialysis sessions that he attended per month, from 12 sessions to about 5 sessions, and decreased in treatment with epoetin alfa (EPREX®) delivered subcutaneously, from 4000 units twice a week to 4000 units once every two weeks.

TABLE 11
Kidney function marker levels in 46-year old patient
suffering from chronic renal failure before and after
therapy with reprogrammed cells.

| Kidney | Pre-therapy | Post-therapy | |
|---|---|---|---|
| Function Marker | Sep. 21, 2008 | Feb. 24, 2009 | Apr. 20, 2009 |
| Hemoglobin [g/dl] | 6.5 | 10.3 | 9.4 |
| TLC/WBC (per μl) | 5500 | 8200 | 10100 |
| PLT (per μl) | 159000 | 246000 | 300000 |
| FBS [mg/dl] | 133 | 120 | 121 |
| 2 hrs PP [mg/dl] | 170 | 172 | 169 |
| AST [U/L] | 9 | 34 | 32 |
| ALT [U/L] | 5 | 22 | 15 |
| Urea [mg/dl] | 109 | 51 | 65 |
| Creatinine [mg/dl] | 7.5 | 4.9 | 4 |
| S. Albumin [g/dl] | 3.5 | 3.7 | 4 |
| PT [seconds] | 12.7 | 12 | 11 |
| INR | 1 | 1 | 1 |
| Calcium [mg/dl] | 10 | 9.9 | 11 |
| Phosphorus [mg/dl] | 6 | 5.3 | 5 |
| Potassium [mmol/L] | 6.2 | 5.1 | 4.7 |
| Magnesium [mg/dl] | 2.3 | 2.4 | 2.8 |

TLC = total leukocyte count;
WBC = white blood cells;
PP = post prandial;
PLT = platelet;
FBS = fasting blood sugar;
AST = aspartate aminotransferase;
ALT = alanine aminotransferase;
PT = pro-thrombin time;
INR = international normalized ratio (for blood coagulation)

### Example 9

### Materials and Methods

Patients with multiple sclerosis were treated with autologous reprogrammed pluripotent stem cells, mesenchymal stem cells, and neurons (target cells). The target cells were through reprogramming apheresed white blood cells until the target cells developed, as indicated by their distinguishing characteristics as described above. The patients were apheresed by processing 2-3 times their total blood volume. The autologous reprogrammed cells were generated through reprogramming in accordance to the invention. The patients were administered the autologous reprogrammed cells via intravenous infusion into the jugular or veins in the arm or thigh.

### Results

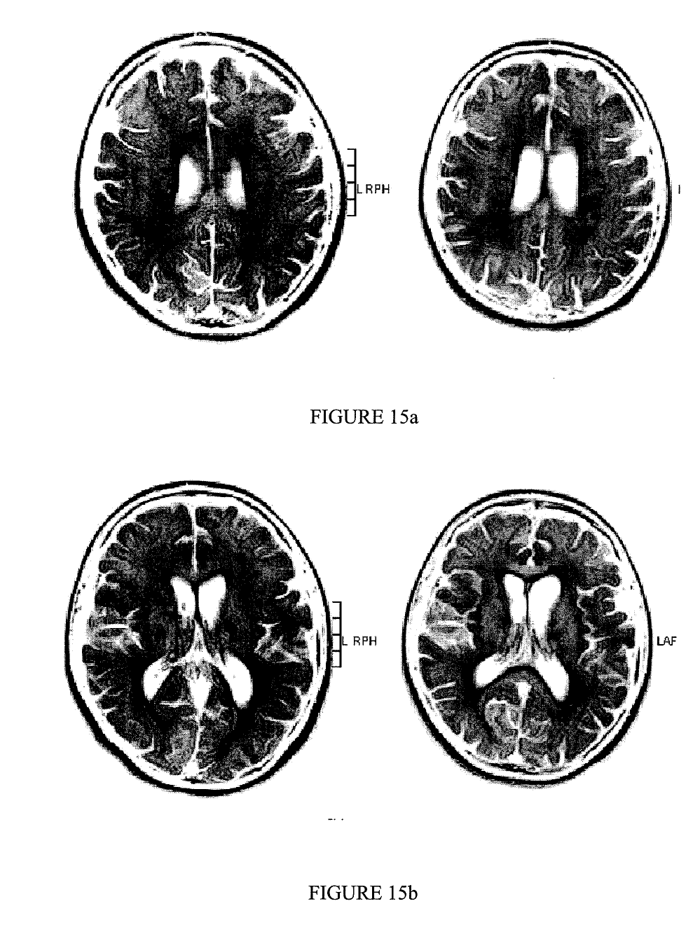

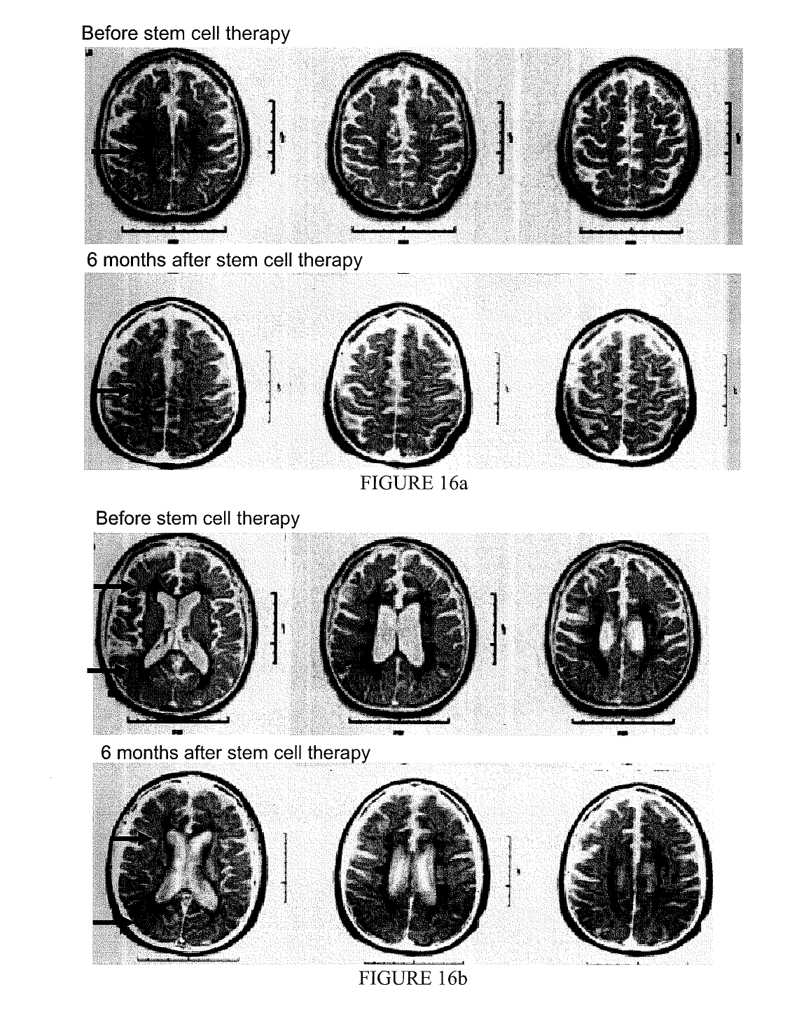

Patients treated with autologous reprogrammed cells showed reduction in lesions in the brain and in the spinal cord. A reduction in lesions can occur within three months of receiving the treatment (FIGS. 15a-b). A reduction in damage to brain tissue occurred within six months (FIGS. 16a-b).

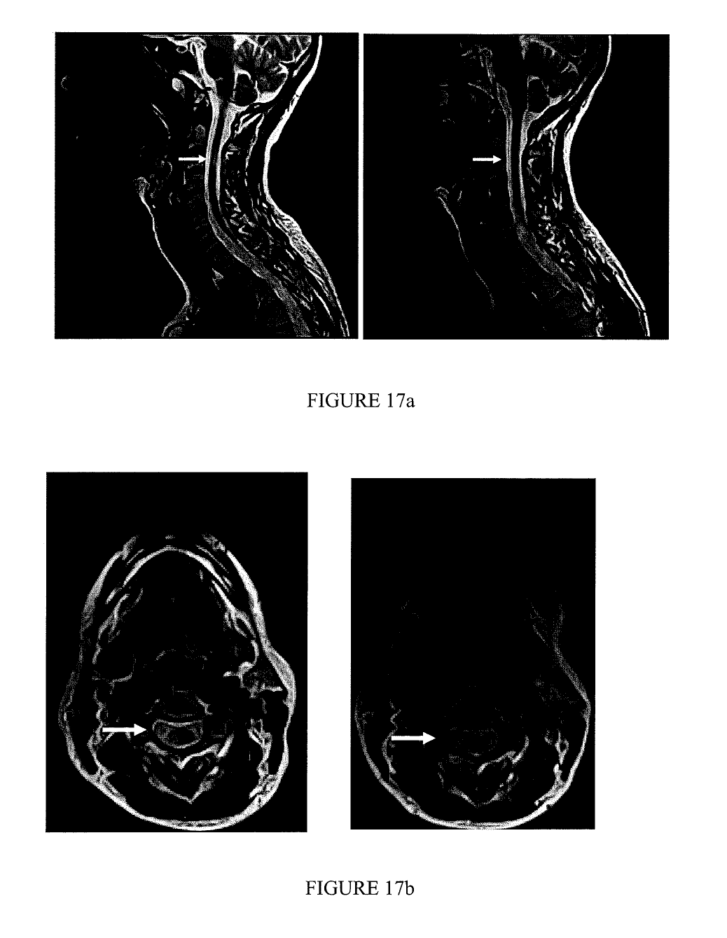

Patients treated with autologous reprogrammed cells also showed removal of spinal cord lesions (FIGS. 17a-b). Further, these patients exhibited improvement in the Kurtzke Expanded Disability Status Scale (EDSS) score, showed remission, and have not participated in conventional therapy up to four years since receiving a single infusion.

### Example 10

### Materials and Methods

A female patient with HIV was treated with autologous reprogrammed haematopoietic stem cells. The patient was apheresed by processing 2-3 times her total blood volume. The target cells were obtained through reprogramming apheresed white blood cells until the target cells developed, as indicated by their distinguishing characteristics as described above. The patient was administered the autologous reprogrammed cells via intravenous infusion into the jugular or veins in the arm or thigh.

### Results

Before treatment, the screening test for HIV-1 and HIV-2 antibodies showed a test value of 3.68. A value of 1.0 or greater is considered positive.

Two months after treatment with autologous reprogrammed cells, the test value for the HIV-1 and. HW-2 antibody screening was 0.46, which indicated that the patient did not show a positive result for HIV-1 and HIV-2. At six months after treatment, the test value for the HW-1 and HW-2 screening was 0.48, further demonstrating that the patient was not showing a positive result for HW.

### Example 11

The effects of treating with autologous reprogrammed cells are demonstrated in patients suffering from other conditions and diseases. The target cells listed herein were obtained through reprogramming apheresed white blood cells until the target cells developed, as indicated by their distinguishing characteristics as described above

### Congestive Heart Failure

A 61-year old male patient suffering from congestive heart failure was infused with autologous reprogrammed cardiomyocytes, pluripotent stem cells, mesenchymal stem cells, and endothelium cells. The treatment led to a improvement in ejection fraction (EF); brain natriuretic peptide precursor levels (Pro BNP), which is a predictor associated with coronary artery disease; blood glucose fasting levels; and HbA1C levels (see Table 12). Furthermore, the heart, which was previously dilated, returned to normal size as evidenced in Table 12 by decrease in end-diastolic left ventricular internal dimension (LVID/D), end-diastolic left ventricular internal dimension (LVID/S), and end-diastolic intraventricular septal thickness (IVSD) as measured by echocardiogram.

TABLE 12
Coronary artery disease markers in a 61-year old patient suffering from congestive
heart failure before and after therapy with reprogrammed cells.

| Coronary | | | | | | |
|---|---|---|---|---|---|---|
| Artery Disease | 1-mo before | 1-mo after | 2-mos after | 4-mos after | 6-mos after | 10-mos after |
| Markers | treatment | treatment | treatment | treatment | treatment | treatment |
| LVID/D [cm] | 6.5 | 6.3 | 5.9 | 6.9 | 6.5 | 5.7 |
| LVID/S [cm] | 6.1 | 5.6 | 5.4 | 5.9 | 5.7 | 4.9 |
| EF | 15 | 18 | 22 | 29 | 33 | 45 |
| IVSD [cm] | 1.3 | 1.3 | 1.2 | 1.2 | 0.6 | 1.0 |
| LVPWD [cm] | 0.7 | 0.6 | 0.6 | 0.8 | 0.6 | 1.0 |
| LV Mass | 283 | 251 | 209.5 | 167 | 151.5 | |
| Index [gm/m] | 141.5 | 133.5 | 111.4 | 88.8 | 81 | |
| Pro BNP | 2969 | 800 | 600 | 600 | 600 | 497 |
| [pg/ml] | | | | | | |
| Blood Glucose | 244 | 118 | 145 | 87 | 95 | 80 |
| Fasting [mg/dl] | | | | | | |
| HbA1C [%] | 9.2 | nd | nd | 7.2 | 6.5 | 6 |

LVID/D = end-diastolic left ventricular internal dimension; LVID/S = end systolic left ventricular internal dimension; EF = ejection fraction; IVSD = end-diastolic intraventricular septal thickness; LVPWD = end-diastolic left ventricular posterior wall thickness; Pro-BNP = precursor of brain natriuretic peptide; HbAlC = glycosylated hemoglobin

### Hepatitis C

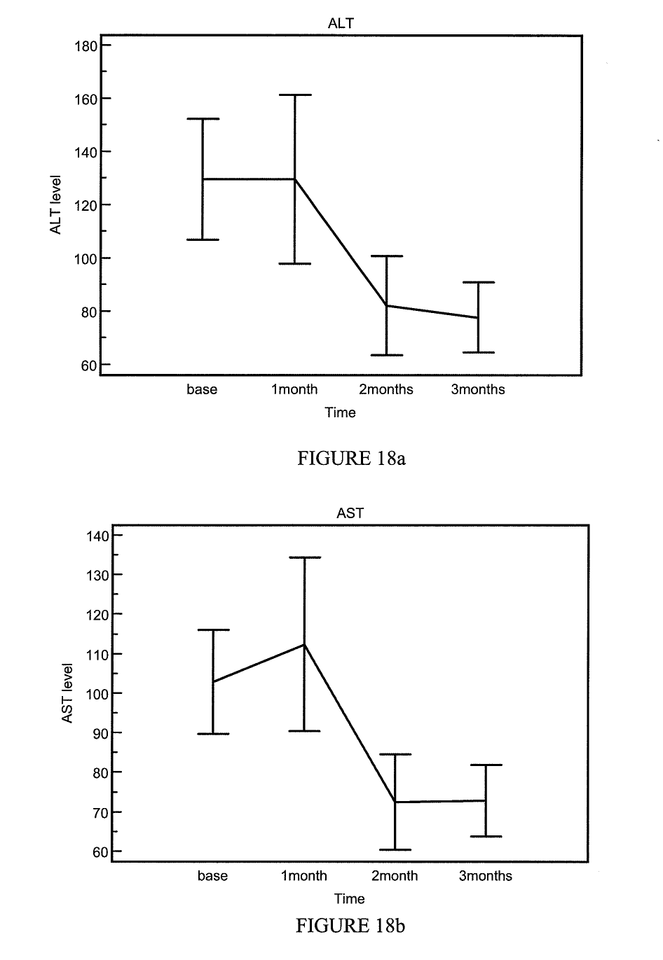

Thirteen patients infected with hepatitis C virus (HCV) and having beta-thalassemia were infused with autologous reprogrammed haematopoietic cells, pluripotent stem cells, mesenchymal stem cells, and hepatocytes. The effects of the treatment revealed a lower or clearance of HCV load (see Table 13), as well as improved blood markers (see Table 14) such as liver enzymes, bilirubin, albumin, pro-thrombin time, and the international normalized ratio (for blood coagulation). In particular, patients experienced normalization of liver enzymes alanine aminotransferase (ALT) and aspartate aminotransferase (AST), which are both associated with inflammation and injury to liver cells (FIGS. 18a-b).

TABLE 13
Viral load in twelve patients infected with hepatitis c
virus and treated with autologous reprogrammed stem cells

| | Baseline | 3 months post-infusion |
|---|---|---|
| Patient ID | viral load × 1000 | viral load × 1000 |
| 1 | 0 | 0 |
| 2 | 164 | 82 |
| 3 | 3 | 0 |
| 4 | 123 | 16 |
| 5 | 16 | 0 |
| 6 | 2 | 0 |
| 6 | 0 | 0 |
| 8 | 7.7 million | 7.7 million |
| 9 | 152 | 106 |
| 10 | 63 | 27 |
| 11 | 387 | 97 |
| 12 | 0 | 0 |
| 13 | 17 | 0 |

TABLE 14
Blood markers in patients infected with hepatitis C virus
and suffering from liver cirrhosis at baseline and after
therapy with reprogrammed cells.

| Blood Markers | Baseline | 3 months post-infusion |
|---|---|---|
| Hemoglobin [g/dl] | 9.8 | 12.9 |
| Platelet (per μl) | 65000 | 120000 |
| WBC (per μl) | 4300 | 7200 |
| Blood sugar (Fasting) [mg/dl] | 210 | 110 |
| Blood 2 hrs (PP) [mg/dl] | 190 | 134 |
| ALT [U/L] | 50 | 24 |
| AST [U/L] | 32 | 31 |
| Bilirubin (Total) [mg/dl] | 6.9 | 3.2 |
| Bilirubin (Direct) [Mg/dm] | 3.1 | 1.8 |
| Serum albumin [g/l] | 1.9 | 3.1 |
| Urea [mg/dl] | 60 | 36 |
| Creatinine [mg/dl] | 1.6 | 1.1 |
| INR | 1.8 | 1.0 |
| PT [seconds] | 18 | 12.8 |

WBC = white blood cells;
PP = post prandial;
ALT = alanine aminotransferase;
AST = aspartate aminotransferase;
INR = international normalized ratio (for blood coagulation);
PT = prothrombin time

For instance, a 44-year old male patient suffering from liver cirrhosis due to hepatitis C virus infection also showed improvement in liver enzymes alanine aminotransferase and aspartate aminotransferase, the international normalized ratio (for blood coagulation), bilirubin, and albumin, as well as random blood sugar (see Table 15). In fact, this patient was on albumin treatment before the therapy with reprogrammed cells, but did not receive albumin after the therapy.

TABLE 15
Blood markers in a 44-year old male patient suffering from liver cirrhosis due to
hepatitis C virus infection at baseline and after therapy with reprogrammed cells.

| | Baseline | Post-therapy | | | |
|---|---|---|---|---|---|
| Blood Marker | Mar. 30, 2009 | Apr. 04, 2009 | Apr. 23, 2009 | May 06, 2009 | Jun. 22, 2009 |
| Hemoglobin | 10.9 | 10.4 | 11.6 | 13.4 | 10.2 |
| [g/dl] | | | | | |
| TLC (per μl) | 2500 | 2700 | 6100 | 4700 | 2800 |
| PLT (per μl) | 20000 | 17000 | 43000 | 50000 | 27000 |
| AST [U/L] | 212 | 170 | 131 | 141 | 120 |
| ALT [U/L] | 62 | 59 | 48 | 51 | 53 |
| Alkaline | 196 | | 147 | | 58 |
| Phosphatase | | | | | |
| [U/L] | | | | | |
| GGT [U/L] | 49 | | 70 | 63 | 35 |
| Bilirubin T | 12.7 | 10.9 | 11.2 | 8.1 | 8.8 |
| [mg/L] | | | | | |
| Bilirubin D | 6.8 | 5.4 | 4.71 | 4.9 | 4 |
| [mg/Dl] | | | | | |
| Urea [mg/dl] | 13 | 18 | 20 | 16 | 25 |
| Creatinine | 0.7 | 0.7 | 0.8 | 0.7 | 0.7 |
| [mg/dl] | | | | | |
| S. Albumin [g/L] | 2.8 | 2.5 | 2.9 | 2.4 | 1.9 |
| RBS [mg/dl] | 212 | 154 | 158 | 188 | 174 |
| INR | 1.9 | 1.8 | 1.72 | 1.69 | 1.72 |
| Potassium | | 3.2 | 3.8 | 4.4 | |
| [mmol/L] | | | | | |
| Sodium | | 134 | 141 | 137 | |
| [mmol/L] | | | | | |
| Uric acid | | 3.5 | | 2.4 | 3 |
| [Mg/dl] | | | | | |
| Alpha Feto | | Normal | | | |
| Protein | | | | | |
| Cryoglobulins | | Negative | | | |

TLC = total leukocyte count;
PLT = platelet;
AST = aspartate aminotransferase;
ALT = alanine aminotransferase;
GGT = gamma-glutamyl transferase;
RBS = random blood sugar;
INR = international normalized ratio (for blood coagulation)

### Head Trauma

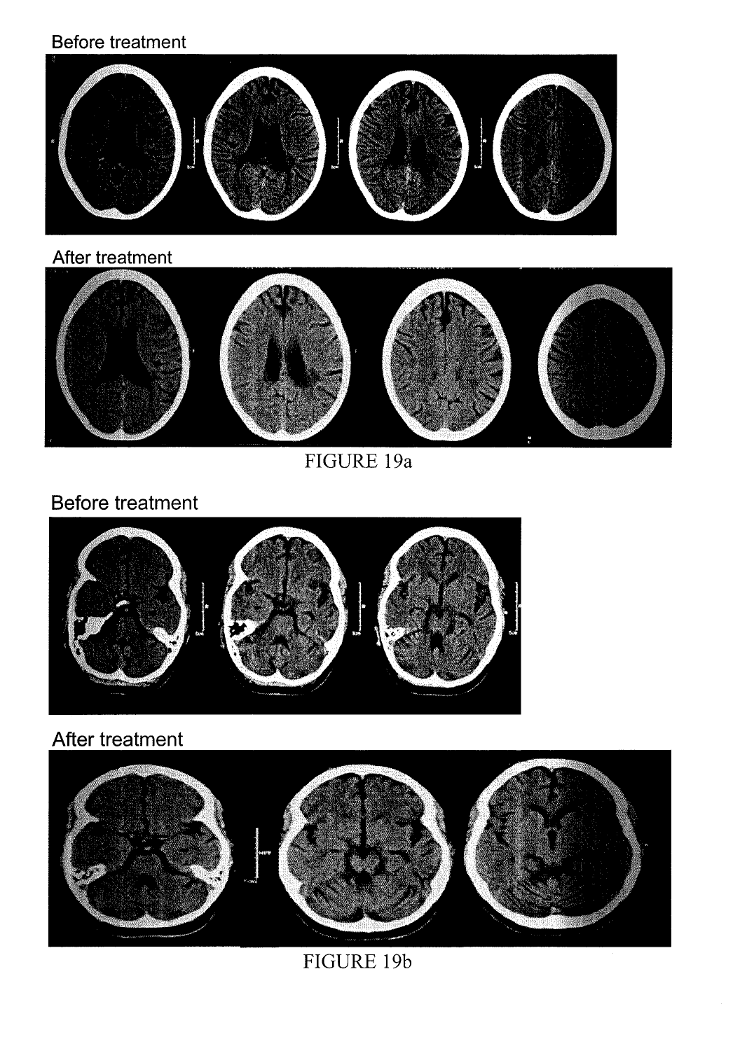

A patient suffering head trauma from a motor accident was treated with an infusion of autologous reprogrammed pluripotent stem cells and neurons. The treatment led to reparation of damaged brain tissue (FIGS. 19a-b), improvement in ejection fraction (EF); brain natriuretic peptide precursor levels (Pro BNP), which is a predictor associated with coronary artery disease; blood glucose fasting levels; and HbA1C levels (see Table 10).

### Lung Disease

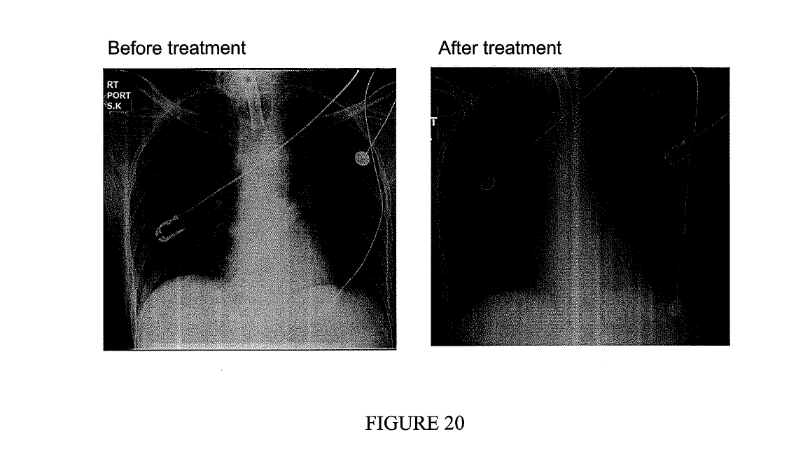

A patient suffering from restrictive lung disease associated with a motor neuron disease was treated with an infusion of autologous reprogrammed pluripotent stem cells, mesenchymal stem cells, alveolar epithelium cells, and endothelium cells. At six months following treatment, forced vital capacity (FVC), which is the volume of air that can forcibly be blown out after full inspiration, increased from 50% to 71%, while forced expiratory volume in 1 second (FEV₁), which is the volume of air that can forcibly be blown out in one second, increased from 64% to 68%. Further, the ratio of FEV₁ to FVC, which is approximately 75-80% in healthy adults, decreased from 100% to 82%. An x-ray scan of the lungs shows less opacity of the lung cavity after treatment (FIG. 20).

### Menopause

A 51-year old patient in menopause was administered an infusion of autologous reprogrammed pluripotent stem cells, pluripotent germ cells, and oocytes. Following the treatment, the patient experienced an increase in various hormone and protein levels, including insulin-like growth factor (IGF-1), esterdiaol, and low-density lipoprotein (LDL) (see Table 16).

TABLE 16
Effects of administering patient in menopause with autologous reprogrammed
stem cells.

| | Pre-therapy | Post-therapy | | |
|---|---|---|---|---|
| Blood Markers | Nov. 23, 2006 | Dec. 09, 2006 | Jan. 01, 2007 | Feb. 17, 2009 |
| IGF-1 [ng/ml] | 91 | 53 | 100 | 143 |
| IGF-1-bind | 5.3 | 4 | 4.5 | 5.9 |
| [mg/L] | | | | |
| GH [μg/L] | 2.4 | 3.5 | 3.5 | 1.5 |
| Progesterone | 1.2 | 1.5 | 17.5 | 2.25 |
| [ng/ml] | | | | |
| Esterdiaol | 26 | 82 | 162 | 168.82 |
| [pg/ml] | | | | |
| Cortisol [μg/dl] | 17.8 | 24 | 4.3 | 24.5 |
| DHEA-S [Nmol/l] | 2.2 | 3.6 | 0.4 | 5.3 |
| Adrenocort | 21.1 | 10 | 10 | |
| [pmol/l] | | | | |
| Tyroglobulin | 47 | 18 | 31 | 1.4 |
| [ng/ml] | | | | |
| Calcitonin | 28 | 40 | 50 | |
| [ng/ml] | | | | |
| FSH [IU/l] | | 41.2 | 5.4 | 3.88 |
| LH [IU/l] | | 15.1 | 1.8 | 6.76 |
| Prolactin [μg/l] | 16.2 | 11.1 | 10.5 | 0.6 |
| Free test [pmol/l] | 8 | 6.4 | 7.5 | |
| Cholest [mg/dl] | 242 | 258 | 280 | 268 |
| HDL [mg/dl] | 70 | 76 | 83 | 63.22 |
| LDL [mg/dl] | 22 | 162 | 173 | 179.9 |
| Triglycer [mg/dl] | 111 | 102 | 122 | 125 |
| Hba1c [%] | 6.5 | 5.3 | 5.5 | 5.4 |
| Fbs [Mg/dl] | 94 | 90 | 102 | 103 |
| Inhibin [U] | 40 | | 30 | |
| 0steocalcin | | | | 6.9 |
| [ng/mL] | | | | |
| TSH [uU/ml] | 0.84 | | 0.7 | 0.5 |
| FT3 [pg/ml] | 3.1 | 2.7 | 2.7 | 1.54 |
| Calcitonin [ng/l] | 28 | 40 | 50 | |
| FT4 [pg/ml] | 1.4 | 0.94 | 0.71 | 0.82 |

IGF-1 = insulin-like growth factor 1;
IGF-1-bind = insulin-like growth factor binding;
GH = growth hormone;
DHEA-S = dehydroepiandrosterone sulfate ester;
Adrenocort = adrenocortical hormone;
FSH = follicle-stimulating hormone;
LH = luteinizing hormone;
HDL = high density lipoprotein;
LDL = low density lipoprotein;
Triglycer = triglyceride;
Hba1C = glycosylated hemoglobin;
Fbs = fetal blood sampling;
TSH = thyroid stimulating hormone;
FT3 = free triiodothryronine;
FT4 = free thyroxine

### Depression

A patient suffering from depression was treated with an infusion of autologous reprogrammed pluripotent stem cells and neurons. Following the treatment, the patient experienced an increase in various hormone and protein levels, including insulin-like growth factor-1 (IGF-1), cortisol, and testosterone (see Table 17).

TABLE 17
Effects of administering patient in menopause with autologous
reprogrammed cells.

| | Pre-therapy | Post-therapy |
|---|---|---|
| Blood Markers | Jun. 11, 2007 | Jul. 23, 2007 |
| GH [μg/l] | <0.05 | 0.06 |
| IGF-1 [ng/ml] | 93.4 | 140 |
| IGF-bp [mg/l] | 4.4 | 5.6 |
| Cortisol [μg/dl] | 4 | 20.9 |
| Adrenocort [pg/ml] | <10 | 17.1 |
| Shbg [nmol/l] | 37.9 | 39.8 |
| FSH [IU/L] | 4.94 | 4.98 |
| LH [IU/L] | 10.1 | 3.72 |
| e2 [pg/ml] | 29 | <28 |
| Testosterone [pmol/l] | 28.4 | 57.8 |
| Prolactin [μg/l] | 15.7 | 1.6 |
| DHEA-S [μmol/l] | 2.3 | 2.9 |
| TSH [μU/ml] | 1.707 | 3.318 |
| FT3 [pg/ml] | 1.69 | 1.87 |
| FT4 [ng/dl] | 0.81 | 0.89 |

GH = growth hormone;
IGF-1 = insulin-like growth factor 1;
IGF-bp = insulin-like growth factor binding protein;
Adrenocort = adrenocortical hormone;
Sh-bg = sex hormone-binding globulin;
FSH = follicle-stimulating hormone;
LH = luteinizing hormone; e2 = estradiol;
DHEA-S = dehydroepiandrosterone sulfate ester;
TSH = thyroid stimulating hormone;
FT3 = free triiodothryronine;
FT4 = free thyroxine

### Non-Obstructive Azoospermia

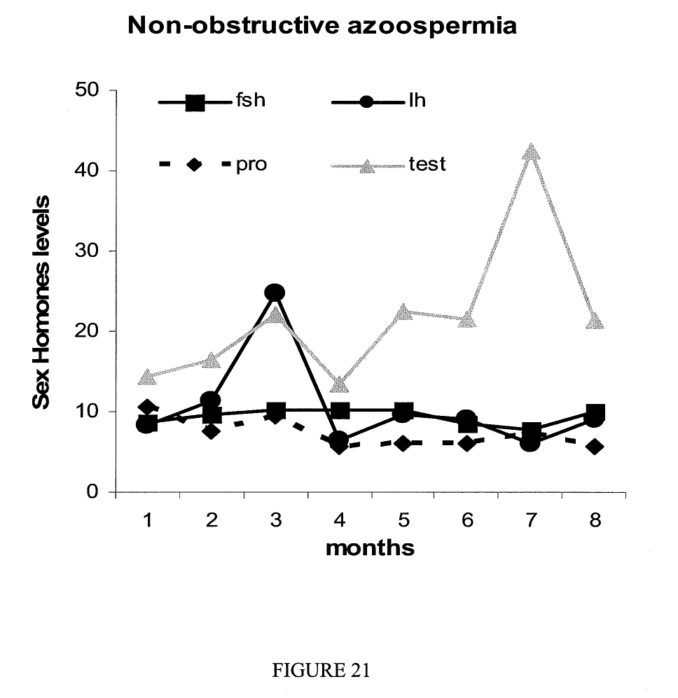

A patient suffering from non-obstructive azoospermia was treated with an infusion of autologous reprogrammed pluripotent stem cells, pluripotent germ cells, and sperm. Following the treatment, the patient experienced an increase in testosterone over a period of eight months (FIG. 21).

### Vision Impairment

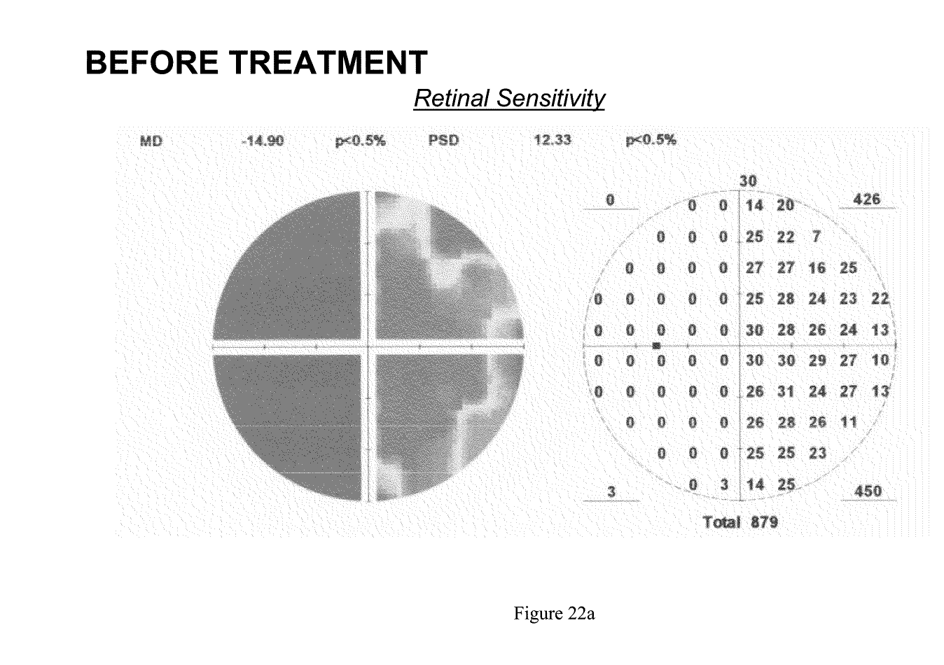

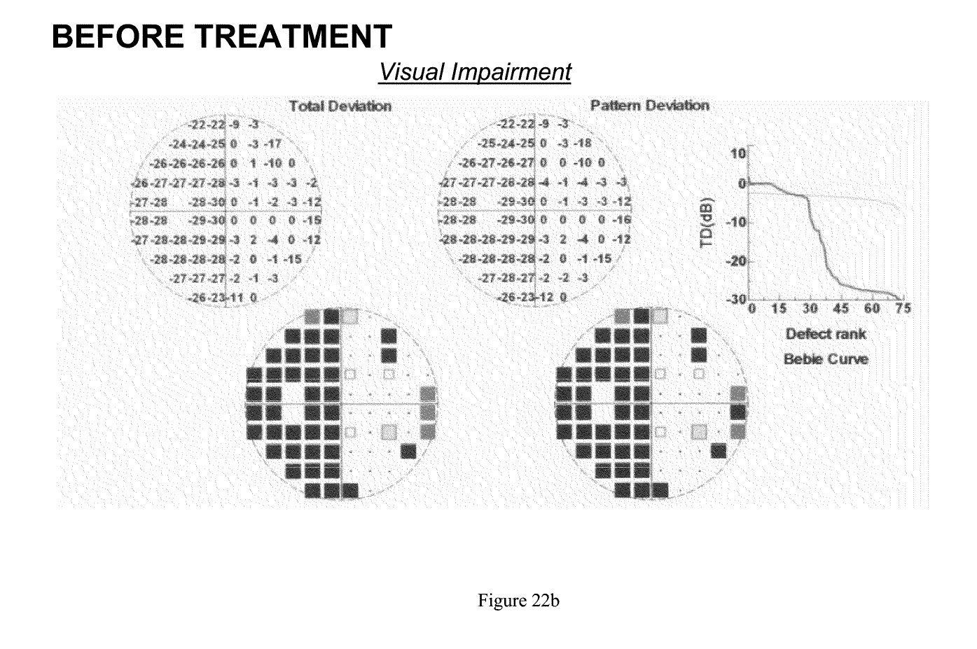

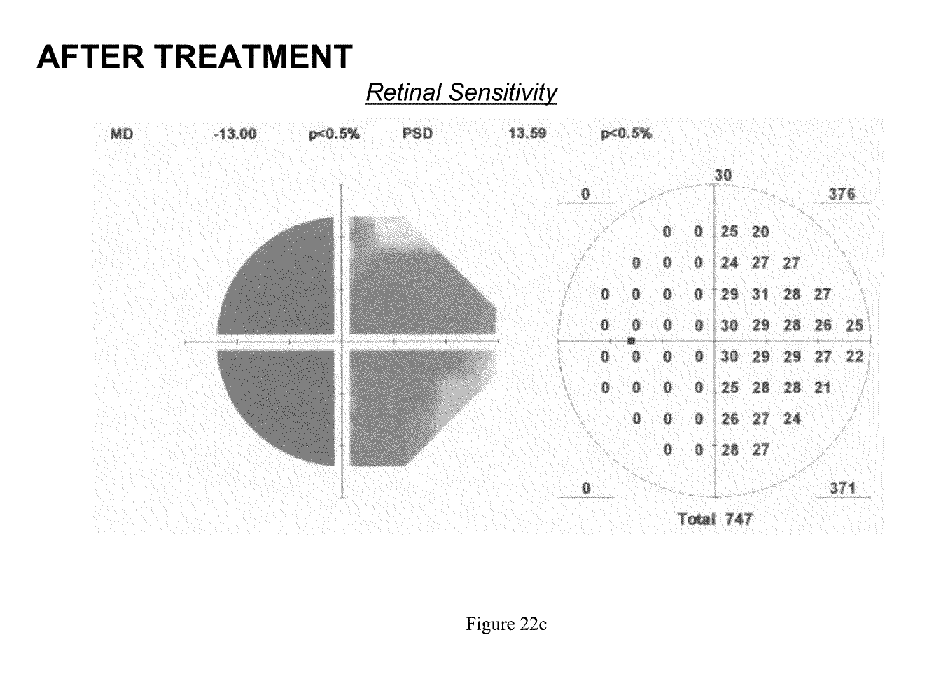

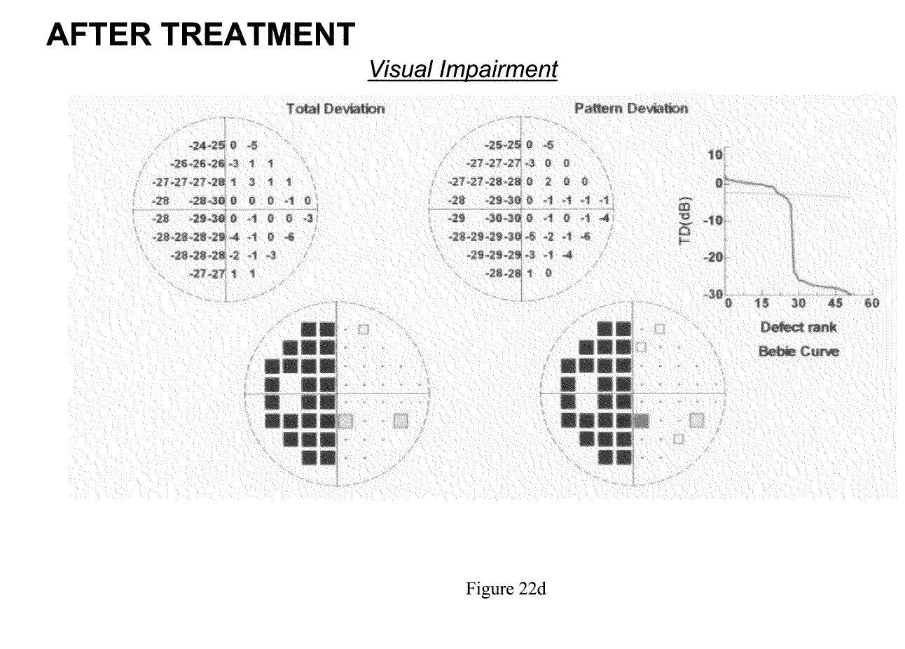

A patient suffering from vision loss due to a benign tumor that had been removed was treated with an infusion of autologous reprogrammed pluripotent stem cells and neurons. Following treatment, the patient experienced increased retinal sensitivity and improvement in vision (FIG. 22).

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method of replenishing tissue or cells of a cell lineage in a patient, comprising (i) obtaining committed cells of a first cell lineage; (ii) reprogramming the committed cells to obtain reprogrammed cells; and (iii) administering the reprogrammed cells to the patient.

2. The method of claim 1, wherein the patient is suffering from a disease or disorder selected from the group consisting of bone marrow failure, haematological conditions, aplastic anemia, beta-thalassemia, motor neuron disease, Parkinson's disease, spinal cord injury, muscular dystrophy, kidney disease, multiple sclerosis, congestive heart failure, hepatitis C virus, human immunodeficiency virus, head trauma, spinal cord injuries, lung disease, depression, non-obstructive azoospermia, andropause, menopause and infertility, rejuvenation, scleroderma ulcers, psoriasis, wrinkles, liver cirrhosis, autoimmune disease, alopecia, retinitis pigmentosa, crystalline dystrophy/blindness, diabetes, liver cirrhosis, and infertility.

3. The method of claim 1, wherein the reprogrammed cells are selected from the group consisting of pluripotent stem cells, haematopoietic stem cells, neuronal stem cells, epithelial stem cells, mesenchymal stem cells, endodermal and neuroectodermal stem cells, germ cells, extraembryonic, embryonic stem cells, kidney cells, alveolar epithelium cells, alveolar endoderm cells, neurons, ectoderm cells, islet cells, acinar cells, oocytes, sperm, hepatocytes, keratinocytes, melanocytes, osteocytes, dermal papilla cells, chondrocytes, adipocytes, endothelium cells, cardiomyocytes, and tropoblasts.

4. The method of claim 1, wherein the committed cells are obtained from whole blood, bone marrow, neuronal tissue, muscle tissue, epidermis or dermis.

5. The method of claim 4, wherein the committed cells are obtained from whole blood.

6. The method of claim 5, wherein the committed cells are obtained through apheresis.

7. The method of claim 5, wherein the committed cells are obtained from mobilized or unmobilized blood.

8. The method of claim 5, wherein the committed cells are selected from the group consisting of include T cells, B cells, eosinophils, basophils, neutrophils, megakaryocytes, monocytes, erythrocytes, granulocytes, mast cells, lymphocytes, leukocytes, and red blood cells.

9. The method of claim 1, wherein reprogramming comprises retrodifferentiation, transdifferentiation, redifferentiation, or a combination thereof, of the committed cells.

10. The method of claim 9, wherein the reprogramming comprises retrodifferentiation of the committed cells to obtain retrodifferentiated cells.

11. The method of claim 10, wherein the committed cells are retrodifferentiated by contacting the committed cells with an agent.

12. The method of claim 11, wherein the committed cells are incubated with the agent.

13. The method of claim 12, wherein the agent engages a receptor that mediates capture, recognition or presentation of an antigen at the surface of the committed cells.

14. The method of claim 11, wherein the receptor is an MHC class I antigen or an MHC class II antigen.

15. The method of claim 11, wherein the agent is an antibody to the receptor.

16. The method of claim 13, wherein the agent is a monoclonal antibody to the receptor.

17. The method of claim 16, wherein the antibody is selected from the group consisting of monoclonal antibody CR3/43 and the monoclonal antibody TAL 1B5.

18. The method of claim 1, wherein the retrodifferentiated cells are administered via injection or implantation.

19. The method of claim 18, wherein the retrodifferentiated cells are administered by parenteral, intramuscular, intravenous, subcutaneous, intraocular, oral, transdermal injection, or injection into spinal fluid.

20. The method of claim 9, wherein the reprogramming comprises transdifferentiation of the committed cells to obtain transdifferentiated cells.

21. The method of claim 20, wherein the committed cells are transdifferentiated by culturing the committed cells in a tissue culture medium comprising one or more retrodifferentiation agents and one or more differentiation promoting agents.

22. The method of claim 21, wherein the tissue culture medium is selected from the group consisting of Iscove's Modified Dulbecco's Medium (IMDM), Dulbecco's Modified Eagle Medium (DMEM), Eagle's minimum essential (EME) medium, alpha-minimum essential medium (α-MEM), Roswell Park Memorial Institute (RPMI; site where medium was developed) 1640, Ham-F-12, E199, MCDB, Leibovitz L-15, and Williams Medium E or any commercially available culture media.

23. The method of claim 21, wherein the differentiation promoting agent is an anticoagulant, cheating agent, or antibiotic.

24. The method of claim 21, wherein the differentiation promoting agent is a vitamin, mineral, or derivative thereof.

25. The method of claim 24, wherein the vitamin, mineral, or derivative thereof is selected from the group consisting of vitamin A, vitamin B₃, vitamin C, vitamin D, vitamin K, retinoic acid, nicotinamide, zinc or zinc compound, and calcium or calcium compounds.

26. The method of claim 21 wherein the differentiation promoting agent is a natural or synthetic hormone.

27. The method of claim 26, wherein the natural or synthetic hormone is hydrocortisone or dexamethasone.

28. The method of claim 21, wherein the differentiation promoting agent is an amino acid or derivative thereof.

29. The method of claim 28, wherein the amino acid or derivative there of is selected from the group consisting of L-glutamine (L-glu), ergothioneine (EGT), proline, and non-essential amino acids (NEAA).

30. The method of claim 21, wherein the differentiation promoting agent is a chemical compound or a derivative thereof.

31. The method of claim 30, wherein the chemical compound or derivative thereof is selected from the group consisting of β-mercaptoethal, dibutyl cyclic adenosine monophosphate (db-cAMP), monothioglycerol (MTG), putrescine, dimethyl sulfoxide (DMSO), hypoxanthine, adenine, forskolin, cilostamide, and 3-isobutyl-1-methylxanthine.

32. The method of claim 21, wherein the differentiation promoting agent is a nucleoside or analogue thereof.

33. The method of claim 32, wherein the nucleoside or analogue thereof is 5-azacytidine.

34. The method of claim 21, wherein the differentiation promoting agent is an acids or salt thereof.

35. The method of claim 34, wherein the acid or salt thereof is selected from the groups consisting of ascorbic acid, pyruvate, okadic acid, linoleic acid, ethylenediaminetetraacetic acid (EDTA), disodium EDTA, ethylene glycol tetraacetic acid (EGTA), Anticoagulant Citrate Dextrose Formula A (ACDA), sodium butyrate, and glycerophosphate.

36. The method of claim 21, wherein the differentiation promoting agent is an antibiotic or drug.

37. The method of claim 36, wherein the antibiotic or drug is selected from the group consisting of G418, gentamycine, Pentoxifylline (1-(5-oxohexyl)-3, 7-dimethylxanthine), and indomethacin.

38. The method of claim 21, wherein the differentiation promoting agent is a protein.

39. The method of claim 38, wherein the protein is tissue plasminogen activator (TPA).

40. The method of claim 21, wherein the tissue culture medium contains autologous plasma; platelets; serum; or sera of mammalian origin.

41. The method of claim 21, wherein the cells are cultured in blood bags, scaffolds, tissue culture bags, or plastic tissue culture vessels.

42. The method of claim 41, wherein the tissue culture vessels are adherent or non-adherent tissue culture vessels.

43. The method of claim 41, wherein the tissue culture vessels are coated or uncoated.

44. The method of claim 43, wherein the tissue culture vessels are coated with an agent selected from the group consisting of gelatin, collagen, matrigel, or extracellular matrix.

45. The method of claim 21, wherein the cells are cultured at a temperature of between about 18 and about 40° C.

46. The method of claim 21, wherein the cells are cultured at a carbon dioxide levels of between about 4 and about 10%.

47. The method of claim 21, wherein the cells are cultured at an oxygen level of between about 10 and about 35%.

48. A method of treating a disease or tissue injury in a patient in need thereof, comprising (i) obtaining committed cells; (ii) reprogramming the committed cells to obtain reprogrammed target cells; and (iii) administering the reprogrammed target cells to the patient.

49. The method of claim 1, wherein the target cells are administered in a pharmaceutical composition.

50. The pharmaceutical composition of claim 49.

51. Use of one or more reprogrammed target cells in a preparation of a medicament or pharmaceutical composition for repairing or replenishing tissue or cells of a cell lineage in a patient, or for treating a disease or tissue injury.

52. A method of obtaining reprogrammed target cells for administration to a patient in need thereof, wherein the method comprises (i) obtaining committed cells of a first cell lineage; and (ii) reprogramming the committed cells to obtain reprogrammed target cells.

53. A pharmaceutical composition comprising reprogrammed target cells obtained by the method of claim 52, and at least one pharmaceutically acceptable excipient.

54. A method of preparing a medicament or pharmaceutical composition for administration to a patient in need thereof, wherein the method comprises: (i) obtaining committed cells of a first cell lineage; (ii) reprogramming the committed cells to obtain reprogrammed target cells; and, (iii) optionally, combining the reprogrammed target cells with one or more pharmaceutical excipients.

55. A pharmaceutical composition prepared by the method of claim 54.
